(19) United States
(12) Patent Application Publication
(10) Pub. No.: US 2026/0347327 A1
(43) Pub. Date: Nov. 5, 2026

(54) Method for Preparing Key Intermediate III for Resmetirom

(71) Applicant: JIANGXI SYNERGY PHARMACEUTICAL CO., LTD., Yichun (CN)

(72) Inventors: Feng YANG, Yichun (CN); Zeyuan PANG, Yichun (CN); Siming YE, Yichun (CN); Wangteng WU, Yichun (CN); Gaoxiang ZHU, Yichun (CN)

(21) Appl. No.: 18/869,747

(22) PCT Filed: Jun. 7, 2024
(86) PCT No.: PCT/CN2024/097886

§ 371 Date: Jul. 10, 2026

(30) Foreign Application Priority Data
Sep. 21, 2023 (CN) 202311220522.8

(51) Int. Cl. C07D 237/14; C07D 407/04

(52) U.S. Cl. CPC C07D 237/14; C07D 407/04

(57) ABSTRACT

Provided is a method for preparing a key intermediate III for Resmetirom, which relates to the technical field of pharmaceutical intermediates. The key intermediate III (formula III) for Resmetirom is prepared from a compound having a structure shown in formula V and a compound having a structure shown in formula E.

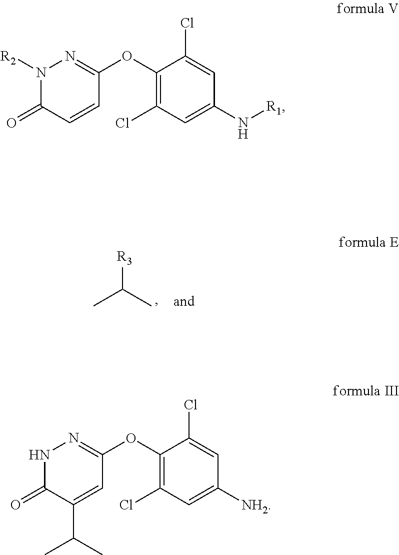

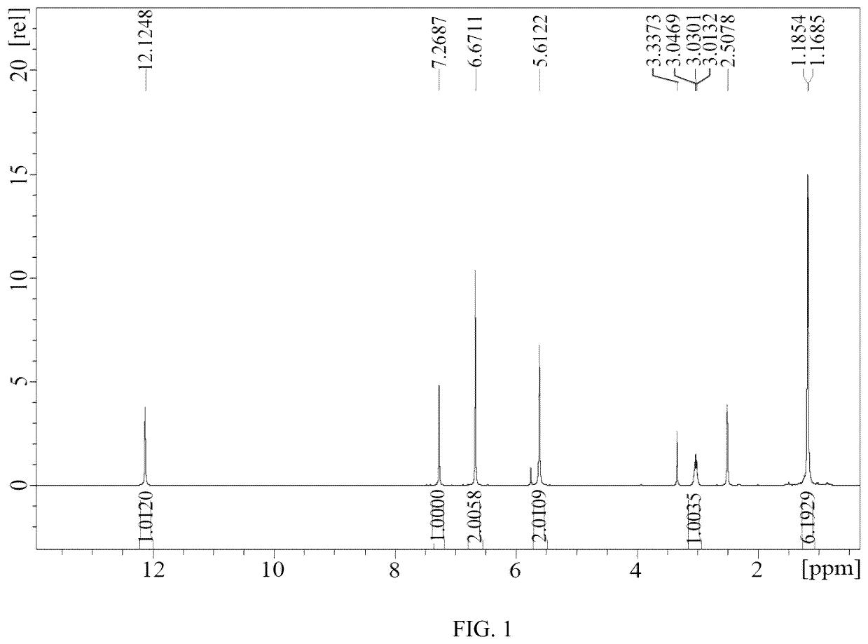

### CROSS REFERENCE TO RELATED APPLICATION

This application is a national stage application of International Patent Application No. PCT/CN2024/097886, filed on Jun. 7, 2024, which claims the priority of a Chinese patent application No. 202311220522.8, entitled “Method for preparing key intermediate III for Resmetirom”, and filed with the China National Intellectual Property Administration on Sep. 21, 2023. The disclosure of the two applications is incorporated by references herein in their entireties as part of the present application.

### TECHNICAL FIELD

The present disclosure relates to the technical field of pharmaceutical intermediates, and in particular to a method for preparing a key intermediate III for Resmetirom.

### BACKGROUND

Resmetirom (CAS: 920509-32-6) is a selective thyroid hormone receptor-β agonist developed by Madrigal Pharmaceuticals. Resmetirom could be used to treat nonalcoholic steatohepatitis. In December 2022, Madrigal Pharmaceuticals, the original new drug research and development company, announced positive top-line results of key phase III clinical trials of Resmetirom. In July 2022, a marketing application of Resmetirom was submitted to the Food and Drug Administration (FDA) of the United States. After being approved for marketing, Resmetirom will become the first marketed drug for treating nonalcoholic steatohepatitis worldwide.

A molecular structure of Resmetirom is shown in the following formula I:

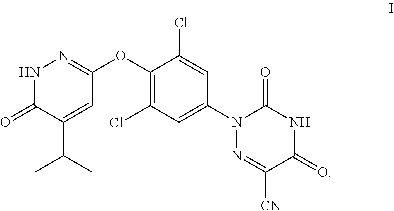 [SMILES: CC(C)c1cc(Oc2c(Cl)cc(-n3nc(C#N)c(=O)[nH]c3=O)cc2Cl)n[nH]c1=O]

Patent publications WO2007009913 and CN112707892A disclose a preparation method of Resmetirom, namely, a first synthesis route as follows:

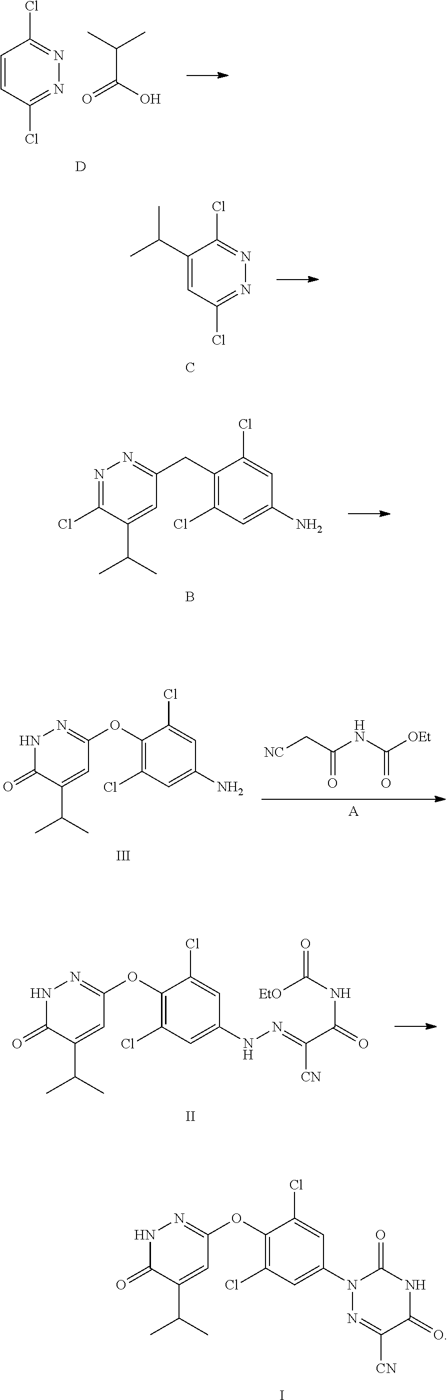

In the first synthesis route, a compound D is reacted with isobutyric acid in the action of silver nitrate and ammonium persulfate to produce a compound C with a yield of 67%. The compound C is subjected to a substitution reaction with 4-amino-2,6-dichlorophenol to produce a compound B with a yield of 53%. The compound B is hydrolyzed to produce a compound III with a yield of 50%. The compound III is subjected to a diazotization reaction, then an addition reaction with a compound A, and then a double bond migration reaction to produce a compound II with a yield of 56%. Finally, the compound II undergoes a ring-closing reaction to obtain a compound I with a yield of 50%.

Patent publications WO2014043706 and CN105008335 disclose another preparation method of Resmetirom, namely, a second synthesis route as follows:

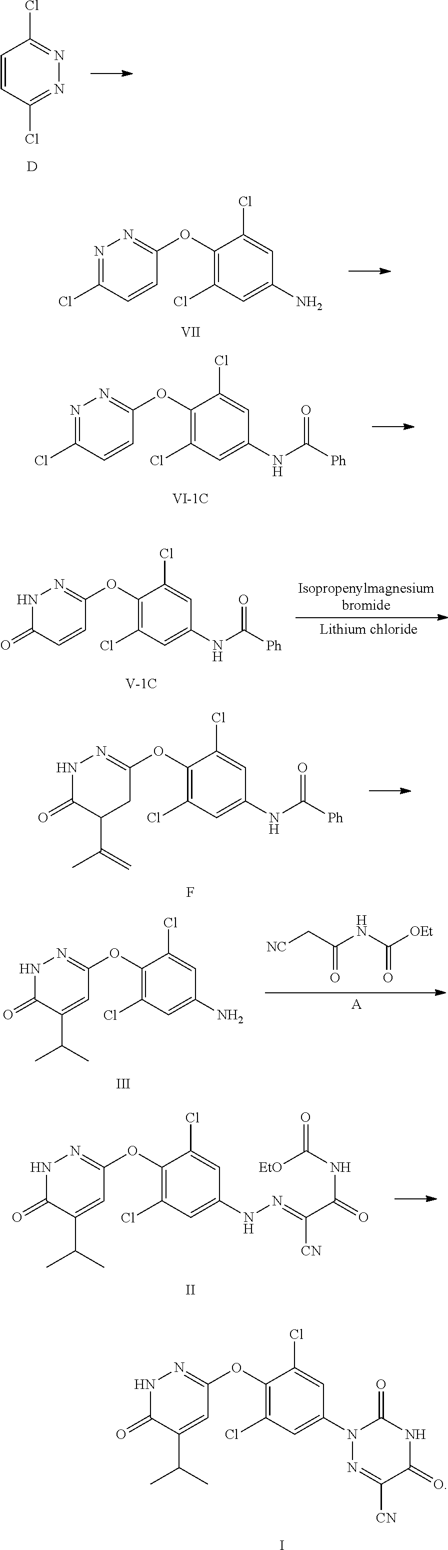

In the second synthesis route, a compound D is subjected to a substitution reaction with 4-amino-2,6-dichlorophenol in the presence of cesium carbonate to produce a compound VII. The compound VII is reacted with benzoic anhydride to produce an amino-protected compound VI-1c. The compound VI-1c is hydrolyzed to produce a compound V-1C. A total yield of the above three reactions is 74%. The compound V-1C is subjected an addition reaction with isopropenylmagnesium bromide (3.5 equivalents) in the presence of lithium chloride (3 equivalents) to produce a compound F. The compound F is subjected to an alkaline hydrolysis reaction and a double bond migration reaction to obtain the compound III with a purity of 87.6%. The compound III is subjected to a diazotization reaction, a double bond migration reaction, and a ring-closing reaction to produce the compound I.

It can be seen from the above routes that the compound III is a key intermediate for the synthesis of Resmetirom. However, in the first synthesis route, for the synthesis of the compound III, each step has a low yield, silver nitrate adopted in the first step is expensive, and the compound C is oily and difficult to purify. The cesium carbonate, isopropylmagnesium bromide, and lithium chloride adopted in the second synthesis route are expensive, and the isopropylmagnesium bromide and lithium chloride are added in large quantities, which results in a large amount of solid and liquid waste and is not conducive to environmental protection. In addition, 4-amino-2,6-dichlorophenol adopted in both the first synthesis route and the second synthesis route has unstable chemical properties and is easily oxidized, which makes purification difficult.

Therefore, there is currently an urgent need to develop a method for preparing a key intermediate III for Resmetirom, which has a high yield, a low cost, easy purification, and excellent environmental friendliness.

### SUMMARY

In view of this, the present disclosure provides a method for preparing a key intermediate III for Resmetirom. In the present disclosure, the key intermediate III is prepared with a compound having a structure shown in formula V and a compound having a structure shown in formula E. The method is simple and has a low cost and a high yield. In addition, intermediate products of the method all are solid, which is convenient for purification and results in prominent environmental friendliness.

To achieve the object of the present disclosure, the present disclosure provides the following technical solutions:

A method for preparing a key intermediate III for Resmetirom is provided, including the following step:
providing a compound having a structure shown in formula V:

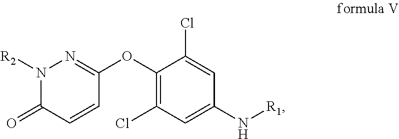

where in formula V, R₁ is H or an amino-protecting group, and the amino-protecting group has 1 to 10 carbon atoms; and R₂ is H or

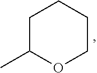

under the condition that R₁ and R₂ in formula V both are H, the method for preparing the key intermediate III for the Resmetirom includes the following step:
(i) mixing the compound having the structure shown in formula V, a compound having a structure shown in formula E, a first alkaline compound, and a first organic solvent, and subjecting a resulting mixture to an addition-elimination-double bond migration reaction, to obtain the key intermediate III for the Resmetirom, the key intermediate III for the Resmetirom having a structural formula shown in formula III:

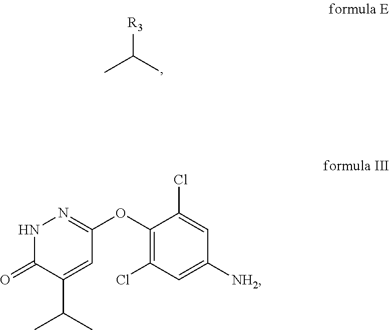

where in formula E, R₃ is selected from the group consisting of —NO₂, —SO₂Ph, —SO₂Me, —CN, —CF₃, and —CCl₃; and
under the condition that R₁ and R₂ in formula V are not both H, the method for preparing the key intermediate III for the Resmetirom includes the following steps:
(1) mixing the compound having the structure shown in formula V, the compound having the structure shown in formula E, a second alkaline compound, and a second organic solvent, and subjecting a resulting mixture to an addition-elimination-double bond migration reaction to obtain a compound having a structure shown in formula IV:

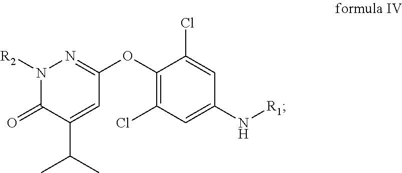

and
(2) removing R₁ and/or R₂ in the compound having the structure shown in formula IV through a deprotection reaction, to obtain the key intermediate III for the Resmetirom.

In some embodiments, the amino-protecting group is selected from the group consisting of t-BuOCO—, PhCO—, CH₃CO—, CF₃CO—, and CCl₃CO—.

In some embodiments, in step (i) and step (1), the alkaline compound is independently at least one selected from the group consisting of 1,8-diazabicyclo[5.4.0]undec-7-ene (DBU), 1,1,3,3-tetramethylguanidine, sodium amide, potassium tert-butoxide, sodium hydride, potassium carbonate, sodium carbonate, KOH, and NaOH, and a molar ratio of the compound having the structure shown in formula V to the alkaline compound independently ranges from 1:0.5 to 1:10;
in step (i) and step (1), a molar ratio of the compound having the structure shown in formula V to the compound having the structure shown in formula E independently ranges from 1:1 to 1:5; and
in step (i) and step (1), the addition-elimination-double bond migration reaction is conducted at a temperature independently of 0° C. to 120° C. for 1 h to 50 h independently.

In some embodiments, under the condition that in formula IV, R₁ is t-BuOCO— and R₂ is H or

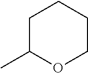

or under the condition that in formula IV, R₁ is H and R₂ is not H, the deprotection reaction in step (2) is an acidic deprotection reaction, and an acid adopted for the acidic deprotection reaction is selected from the group consisting of a solution of hydrogen chloride in ethyl ether, a solution of hydrogen chloride in tetrahydrofuran, and a solution of hydrogen chloride in 1,4-dioxane; and the acidic deprotection reaction is conducted at a temperature of 0° C. to 100° C.

In some embodiments, under the condition that in formula IV, R₁ is not H or t-BuOCO— and R₂ is H, the deprotection reaction in step (2) is an alkaline hydrolysis reaction, and an alkali adopted for the alkaline hydrolysis reaction is sodium hydroxide or potassium hydroxide; a molar ratio of the compound having the structure shown in formula IV to the alkali ranges from 1:2 to 1:10; and a solvent adopted for the alkaline hydrolysis reaction is an organic solvent-water mixed solvent, and an organic solvent in the organic solvent-water mixed solvent is at least one selected from the group consisting of ethanol, methanol, tetrahydrofuran, 1,4-dioxane, and dimethyl sulfoxide (DMSO).

In some embodiments, under the condition that in formula IV, neither R₁ or R₂ is H and R₁ is not t-BuOCO—, the deprotection reaction in step (2) includes an acidic deprotection reaction and an alkaline hydrolysis reaction that are conducted sequentially; an acid adopted for the acidic deprotection reaction is one selected from the group consisting of a solution of hydrogen chloride in ethyl ether, a solution of hydrogen chloride in tetrahydrofuran, and a solution of hydrogen chloride in 1,4-dioxane; the acidic deprotection reaction is conducted at a temperature of 0° C. to 100° C.; an alkali adopted for the alkaline hydrolysis reaction is sodium hydroxide or potassium hydroxide; a molar ratio of the compound having the structure shown in formula IV to the alkali ranges from 1:2 to 1:10; and a solvent adopted for the alkaline hydrolysis reaction is an organic solvent-water mixed solvent, and an organic solvent in the organic solvent-water mixed solvent is at least one selected from the group consisting of ethanol, methanol, tetrahydrofuran, 1,4-dioxane, and DMSO.

In some embodiments, under the condition that in formula V, R₁ is not H and R₂ is H, the compound having the structure shown in formula V is prepared by a process including the following steps:
mixing a compound having a structure shown in formula D, a compound having a structure shown in formula G, a third alkaline compound, and a third organic solvent, and subjecting a resulting mixture to a substitution reaction, to obtain a compound having a structure shown in formula VI:

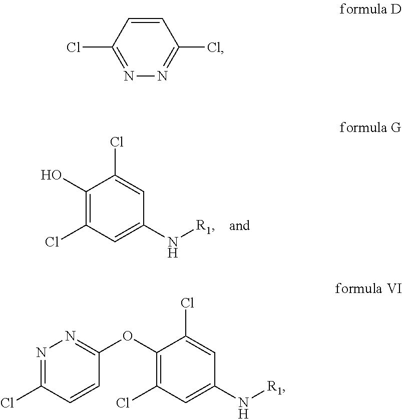

wherein R₁ in formula G and R₁ in formula VI are of the same type as R₁ in formula V; and
mixing the compound having the structure shown in formula VI, acetic acid, and sodium acetate, and subjecting a resulting mixture to a substitution-hydrolysis reaction, to obtain the compound having the structure shown in formula V, which has a structural formula shown in formula V-1:

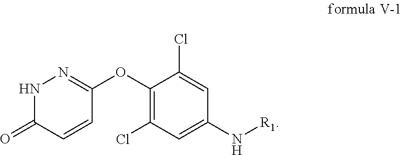

under the condition that in formula V, R₁ is not H and R₂ is not H, the compound having the structure shown in formula V is prepared by a process including the following step:
mixing a compound having a structure shown in formula V-1, 3,4-dihydro-2H-pyran, an acid, and an organic solvent, and subjecting a resulting mixture to an amide protection reaction, to obtain the compound having the structure shown in formula V, which has a structural formula shown in formula V-2:

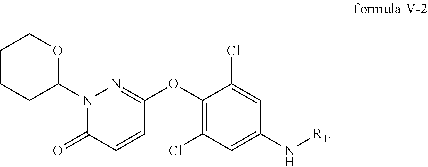

under the condition that in formula V, R₁ is H and and R₂ is H, the compound having the structure shown in formula V is prepared by a process including:
mixing a compound having a structure shown in formula V-1, a fourth alkaline compound, a fourth organic solvent, and water, and subjecting a resulting mixture to a first hydrolysis reaction, to obtain the compound having the structure shown in formula V, which has a structural formula shown in formula V-3:

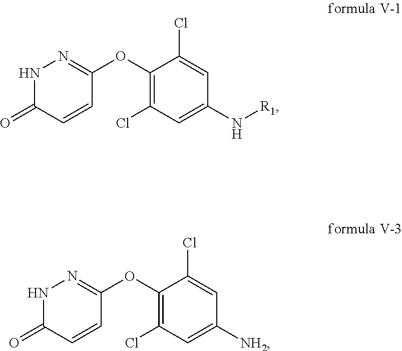

wherein in formula V-1, R₁ is selected from the group consisting of PhCO—, CF₃CO—, CCl₃CO—, and CH₃CO—.
under the condition that in formula V, R₁ is H and R₂ is not H, the compound having the structure shown in formula V is prepared by a process including the following step:
mixing a compound having a structure shown in formula V-2, a fifth alkaline compound, a fifth organic solvent, and water, and subjecting a resulting mixture to a second hydrolysis reaction, to obtain the compound having the structure shown in formula V, which has a structural formula shown in formula V-4:

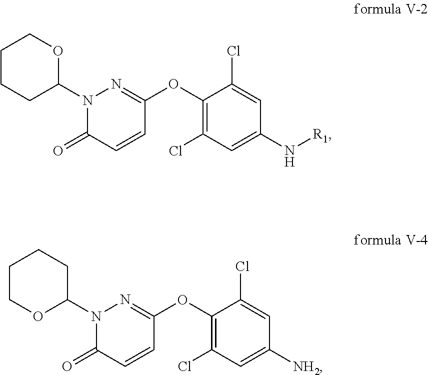

wherein in formula V-2, R₁ is selected from the group consisting of PhCO—, CF₃CO—, CCl₃CO—, and CH₃CO—.

In some embodiments, a molar ratio of the compound having the structure shown in formula D to the compound having the structure shown in formula G ranges from 1:1 to 1:2;
the third alkaline compound is at least one selected from the group consisting of an alkali metal carbonate, an alkali metal hydroxide, and an organic base, and a molar ratio of the compound having the structure shown in formula D to the third alkaline compound ranges from of 1:1 to 1:3;
the third organic solvent adopted for the substitution reaction is at least one selected from the group consisting of N,N-dimethylformamide (DMF), N,N-dimethylacetamide (DMAc), DMSO, N-methylpyrrolidone (NMP), acetonitrile, and 1,4-dioxane, and the substitution reaction is conducted at a temperature of 40° C. to 120° C. for 5 h to 50 h;
a molar ratio of the compound having the structure shown in formula VI to the sodium acetate ranges from 1:1 to 1:5, and a molar ratio of the compound having the structure shown in formula VI to the acetic acid ranges from 1:10 to 1:50; and
the substitution-hydrolysis reaction is conducted at a temperature of 60° C. to 150° C. for 2 h to 20 h.

In some embodiments, in the amide protection reaction, a molar ratio of the compound having the structure shown in formula V-1 to the acid ranges from 1:0.05 to 1:0.5, and a molar ratio of the compound having the structure shown in formula V-1 to the 3,4-dihydro-2H-pyran ranges from 1:1 to 1:4;
the organic solvent adopted for the amide protection reaction is at least one selected from the group consisting of tetrahydrofuran, ethyl acetate, dichloromethane, acetonitrile, acetone, and 1,4-dioxane; and
the amide protection reaction is conducted at a temperature of 30° C. to 80° C. for 1 h to 10 h.

In some embodiments, in the first hydrolysis reaction, a molar ratio of the compound having the structure shown in formula V-1 to the fourth alkaline compound ranges from 1:2 to 1:6, and the fourth alkaline compound is an alkali metal hydroxide;
the fourth organic solvent adopted for the first hydrolysis reaction is at least one selected from the group consisting of ethanol, methanol, tetrahydrofuran, and DMSO; and
the first hydrolysis reaction is conducted at a temperature of 50° C. to 120° C. for 1 h to 10 h.

In the second hydrolysis reaction, a molar ratio of the compound having the structure shown in formula V-2 to the fifth alkaline compound ranges from 1:2 to 1:6, and the fifth alkaline compound is an alkali metal hydroxide;
the fifth organic solvent adopted for the second hydrolysis reaction is at least one selected from the group consisting of ethanol, methanol, tetrahydrofuran, and DMSO; and
the second hydrolysis reaction is conducted at a temperature of 50° C. to 120° C. for 1 h to 10 h.

The present disclosure provides a method for preparing a key intermediate III for Resmetirom. Under the condition that R₁ and R₂ in formula V both are H, the compound having the structure shown in formula V, the compound having the structure shown in formula E, the alkaline compound, and the organic solvent are mixed, and a resulting mixture is subjected to an addition-elimination-double bond migration reaction to directly obtain the key intermediate III for Resmetirom. Under the condition that R₁ and R₂ in formula V are not both H, the compound having the structure shown in formula V, the compound having the structure shown in formula E, the alkaline compound, and the organic solvent are mixed, and a resulting mixture is subjected to an addition-elimination-double bond migration reaction to obtain a compound having a structure shown in formula IV; and R₁ and/or R₂ are removed through a deprotection reaction to obtain the key intermediate III. The method for preparing a key intermediate III according to the present disclosure involves simple steps, easy operations, and a high product yield and does not require expensive silver nitrate, cesium carbonate, isopropenylmagnesium bromide, and lithium chloride. Also, intermediate products in the method all are solid, which is convenient for purification. The method according to the present disclosure leads to a small amount of solid and liquid waste, and is conducive to environmental protection. Further, the compound having the structure shown in formula G adopted in the present disclosure has stable properties and is not easily oxidized. In summary, the method according to the present disclosure could significantly reduce the difficulty and cost of production of the key intermediate III, and is conducive to industrial production.

### BRIEF DESCRIPTION OF THE DRAWING

FIGURE shows an ¹H nuclear magnetic resonance spectrum of the key intermediate III for Resmetirom prepared in Example 20 of the present disclosure.

### DETAILED DESCRIPTION OF THE EMBODIMENTS

The present disclosure provides a method for preparing a key intermediate III for Resmetirom, including the following step:
providing a compound having a structure shown in formula V,

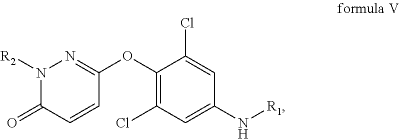

wherein in formula V, R₁ is H or an amino-protecting group, and the amino-protecting group has 1 to 10 carbon atoms; and R₂ is H or

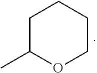

Under the condition that R₁ and R₂ in formula V both are H, the method for preparing the key intermediate III for Resmetirom includes the following step:
(i) mixing the compound having the structure shown in formula V, a compound having a structure shown in formula E, a first alkaline compound, and a first organic solvent, and subjecting a resulting mixture to an addition-elimination-double bond migration reaction, to obtain the key intermediate III for Resmetirom, the key intermediate III for Resmetirom having a structural formula shown in formula III,

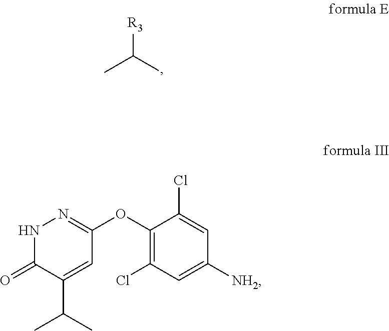

wherein in formula E, R₃ is selected from the group consisting of —NO₂, —SO₂Ph, —SO₂Me, —CN, —CF₃, and —CCl₃.

Under the condition that R₁ and R₂ in formula V are not both H, the method for preparing the key intermediate III for Resmetirom includes the following steps:
(1) mixing the compound having the structure shown in formula V, a compound having the structure shown in formula E, a second alkaline compound, and a second organic solvent, and subjecting a resulting mixture to an addition-elimination-double bond migration reaction, to obtain a compound having a structure shown in formula IV,

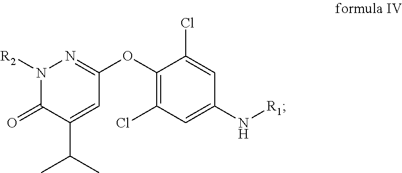

and
(2) removing R₁ and/or R₂ in the compound having the structure shown in formula IV through a deprotection reaction, to obtain the key intermediate III for Resmetirom.

The present disclosure first provides the compound having the structure shown in formula V. In the present disclosure, under the condition that R₁ in formula V is an amino-protecting group, the amino-protecting group has preferably 2 to 8 carbon atoms, and specifically, the amino-protecting group is selected from the group consisting of t-BuOCO—, PhCO—, CH₃CO—, CF₃CO—, and CCl₃CO—.

In some embodiments of the present disclosure, under the condition that in formula V, R₁ is not H and R₂ is H, the method for preparing the intermediate for Resmetirom includes the following steps:
mixing a compound having a structure shown in formula D, a compound having a structure shown in formula G, a third alkaline compound, and a third organic solvent, and subjecting a resulting mixture to a substitution reaction, to obtain a compound having a structure shown in formula VI,

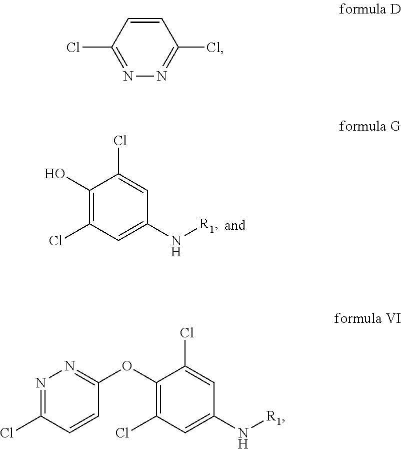

wherein R₁ in formula G and R₁ in formula VI are of the same type as R₁ in formula V;
mixing the compound having the structure shown in formula VI, acetic acid, and sodium acetate, and subjecting a resulting mixture to a substitution-hydrolysis reaction, to obtain the compound having the structure shown in formula V, which has a structural formula shown in formula V-1:

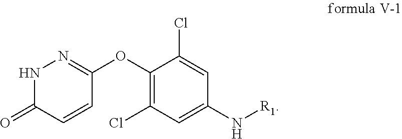

In some embodiments, under the condition that R₁ is not H and R₂ is H, the compound having the structure shown in formula V is synthesized according to a synthesis route as follows:

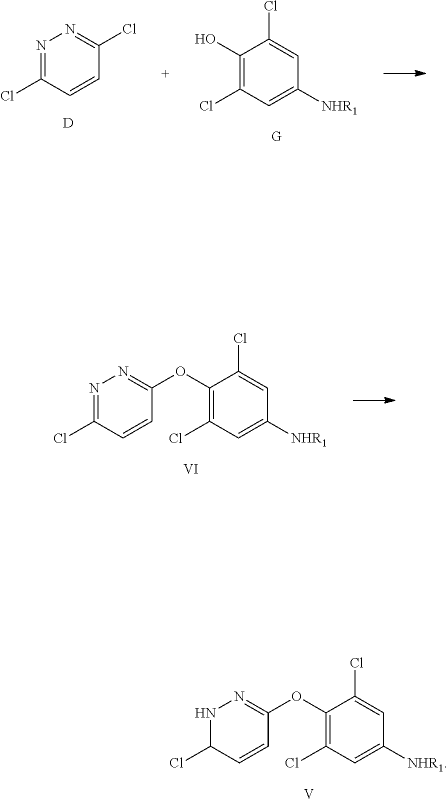

In the present disclosure, a compound having a structure shown in formula D, a compound having a structure shown in formula G, a third alkaline compound, and a third organic solvent are mixed, and a resulting mixture to subjected to a substitution reaction, to obtain a compound having a structure shown in formula VI. In some embodiments of the present disclosure, a molar ratio of the compound having the structure shown in formula D to the compound having the structure shown in formula G is in a range of 1:(1-2) and preferably 1:(1-1.3). In some embodiments, the third alkaline compound is at least one selected from the group consisting of an alkali metal carbonate, an alkali metal hydroxide, and an organic base. In some embodiments, the alkali metal carbonate is at least one selected from the group consisting of potassium carbonate, sodium carbonate, and cesium carbonate. In some embodiments, the alkali metal hydroxide is potassium hydroxide and/or sodium hydroxide. In some embodiments, the organic base is at least one selected from the group consisting of sodium methoxide, sodium ethoxide, and potassium tert-butoxide. In specific embodiments of the present disclosure, the third alkaline compound is potassium carbonate. In some embodiments, a molar ratio of the compound having the structure shown in formula D to the third alkaline compound is in a range of 1:(1-3) and preferably 1:(1-2). In some embodiments, the third organic solvent adopted for the substitution reaction is at least one selected from the group consisting of DMF, DMAC, DMSO, NMP, acetonitrile, and 1,4-dioxane, and is preferably DMF.

In some embodiments of the present disclosure, the substitution reaction is conducted at a temperature of 40° C. to 120° C. and preferably 50° C. to 90° C. In some embodiments, the substitution reaction is conducted for 5 h to 50 h and preferably 10 h to 40 h.

In some embodiments of the present disclosure, a substitution reaction solution produced from the substitution reaction is subjected to a post-treatment. In some embodiments, the post-treatment includes: mixing the substitution reaction solution with water to obtain a mixed solution, adjusting the mixed solution to a pH value of 11, stirring at room temperature, and filtering to obtain a solid product, and washing the solid product with water and then oven-drying to obtain the compound having the structure shown in formula VI. In some embodiments, a reagent adopted for adjusting the pH value of the mixed solution is liquid caustic soda. In some embodiments, the stirring at room temperature is conducted for 30 min.

In the present disclosure, after the compound having the structure shown in formula VI is obtained, the compound having the structure shown in formula VI, acetic acid, and sodium acetate are mixed, and a resulting mixture is subjected to a substitution-hydrolysis reaction to obtain the compound having the structure shown in formula V, which has a structural formula shown in formula V-1. In some embodiments of the present disclosure, a molar ratio of the compound having the structure shown in formula VI to the sodium acetate is in a range of 1: (1-5) and preferably 1:(1.5-3). In some embodiments, a molar ratio of the compound having the structure shown in formula VI to the acetic acid is in a range of 1:(10-50) and preferably 1:(15-30). In some embodiments, the acetic acid is glacial acetic acid. In some embodiments, the substitution-hydrolysis reaction is conducted at a temperature of 60° C. to 150° C. and preferably 90° C. to 120° C. In some embodiments, the substitution-hydrolysis reaction is conducted for 2 h to 20 h and preferably 5 h to 15 h.

In some embodiments of the present disclosure, after the substitution-hydrolysis reaction, a substitution-hydrolysis reaction solution produced from the substitution-hydrolysis reaction is subjected to a post-treatment. In some embodiments, the post-treatment includes: subjecting the substitution-hydrolysis reaction solution to rotary evaporation under reduced pressure until dryness to obtain a residue, mixing the residue and water, stirring a resulting mixture at room temperature, and filtering to obtain a solid product; and washing the solid product with water and then drying to obtain the compound having the structure shown in formula V (formula V-1). In some embodiments, the stirring at room temperature is conducted for 2 h.

In some embodiments of the present disclosure, under the condition that in formula V, R₁ is not H and R₂ is not H, the key intermediate for Resmetirom having the structure shown in formula V is prepared by a process including the following step:
mixing a compound having a structure shown in formula V-1, 3,4-dihydro-2H-pyran, an acid, and an organic solvent, and subjecting a resulting mixture to an amide protection reaction, to obtain the compound having the structure shown in formula V, which has a structural formula shown in formula V-2,

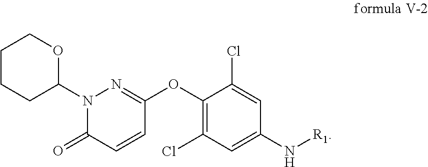

In some embodiments, under the condition that R₁ is not H, and R₂ is not H, a synthesis route of the compound having the structure shown in formula V is as follows:

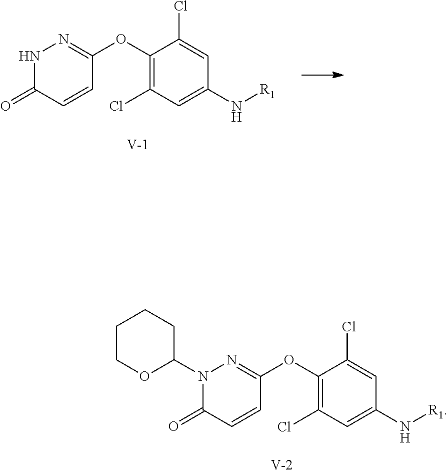

In the present disclosure, a preparation process of the compound having the structure shown in formula V-1 is consistent with those described in the above solutions, and will not be repeated here. In some embodiments, the acid is an organic acid, preferably at least one selected from the group consisting of p-toluenesulfonic acid, methanesulfonic acid, and trifluoroacetic acid, and further more preferably p-toluenesulfonic acid. In some embodiments, a molar ratio of the compound having the structure shown in formula V-1 to the acid is in a range of 1:(0.05-0.5) and preferably 1:(0.05-0.3). In some embodiments, a molar ratio of the compound having the structure shown in formula V-1 to the 3,4-dihydro-2H-pyran is in a range of 1:(1-4) and preferably 1:(1-2). In some embodiments, the organic solvent adopted for the amide protection reaction is at least one selected from the group consisting of tetrahydrofuran, ethyl acetate, dichloromethane, acetonitrile, acetone, and 1,4-dioxane, and preferably tetrahydrofuran. In some embodiments, the amide protection reaction is conducted at a temperature of 30° C. to 80° C. and preferably 40° C. to 70° C. In some embodiments, the amide protection reaction is conducted for 1 h to 10 h and preferably 3 h to 8 h.

In some embodiments of the present disclosure, after the amide protection reaction, an amide protection reaction solution produced from the amide protection reaction is subjected to a post-treatment. In some embodiments, the post-treatment includes: cooling the amide protection reaction solution to room temperature and then mixing with water to obtain a mixed solution, adjusting the mixed solution to a pH value of 9, stirring at room temperature, subjecting a resulting mixture to rotary evaporation to remove the organic solvent to obtain a residue, and then filtering the residue to obtain a solid product, and washing the solid product and then drying to obtain the compound having the structure shown in formula V (formula V-2). In some embodiments, a reagent adopted for adjusting the pH value of the mixed solution is potassium carbonate. In some embodiments, the stirring at room temperature is conducted for 30 min. In some embodiments, a reagent adopted for the washing is a mixed solvent of ethyl acetate and heptane. In some embodiments, a volume ratio of the ethyl acetate to the heptane in the mixed solvent is 1:2.

In some embodiments of the present disclosure, under the condition that in formula V, R₁ is H and R₂ is H, the key intermediate for Resmetirom having the structure shown in formula V is prepared by a process including the following step:
mixing a compound having a structure shown in formula V-1, a fourth alkaline compound, a fourth organic solvent, and water, and subjecting a resulting mixture to a first hydrolysis reaction, to obtain the compound having the structure shown in formula V, which has a structural formula shown in formula V-3:

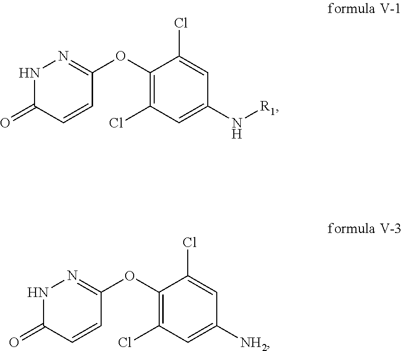

wherein in formula V-1, R₁ is selected from the group consisting of PhCO—, CF₃CO—, CCl₃CO—, and CH₃CO—.

In some embodiments, under the condition that R₁ is H and R₂ is H, a synthesis route of the compound having the structure shown in formula V is as follows:

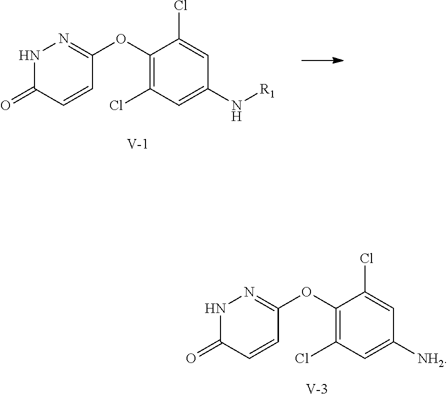

In the present disclosure, a specific preparation process of the compound having the structure shown in formula V-1 is the same as those described in the above solutions, and will not be repeated here.

In some embodiments of the present disclosure, the fourth alkaline compound is an alkali metal hydroxide. In some embodiments, the alkali metal hydroxide is potassium hydroxide and/or sodium hydroxide. In some embodiments, a molar ratio of the compound having the structure shown in formula V-1 to the fourth alkaline compound is in a range of 1:(2-6) and preferably 1:(2.5-4). In some embodiments, the fourth organic solvent adopted for the first hydrolysis reaction is at least one selected from the group consisting of ethanol, methanol, tetrahydrofuran, and DMSO, and preferably ethanol or methanol. In some embodiments, a volume ratio of the fourth organic solvent to the water is in a range of (1-4):1. In some embodiments, the first hydrolysis reaction is conducted at a temperature of 50° C. to 120° C. and preferably 60° C. to 100° C. In some embodiments, the first hydrolysis reaction is conducted for 1 h to 10 h and preferably 3 h to 8 h.

In some embodiments of the present disclosure, after the first hydrolysis reaction, a first hydrolysis reaction solution produced from the first hydrolysis reaction is subjected to a post-treatment. In some embodiments, the post-treatment includes the following steps: cooling the first hydrolysis reaction solution to room temperature, then adjusting with hydrochloric acid to a pH value of 6, subjecting a resulting mixture to rotary evaporation to remove the fourth organic solvent and obtain a residue, and filtering the residue to obtain a solid product, and washing the solid product with water and then drying to obtain the compound having the structure shown in formula V (formula V-3).

In some embodiments of the present disclosure, under the condition that in formula V, R₁ is H and R₂ is not H, the intermediate for Resmetirom shown in formula V is prepared by a process including the following step:
mixing a compound having a structure shown in formula V-2, a fifth alkaline compound, a fifth organic solvent, and water, and subjecting a resulting mixture to a second hydrolysis reaction, to obtain the compound having the structure shown in formula V, which has a structural formula shown in formula V-4:

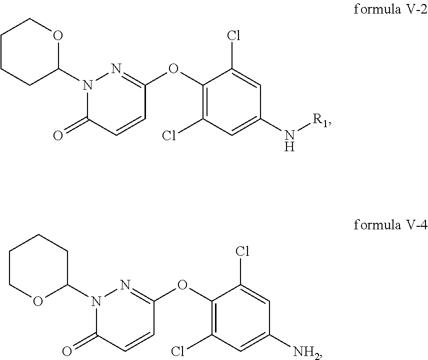

wherein in formula V-2, R₁ is selected from the group consisting of PhCO—, CF₃CO—, CCl₃CO—, and CH₃CO—.

In some embodiments, under the condition that R₁ is H and R₂ is not H, a synthesis route of the compound having the structure shown in formula V is as follows:

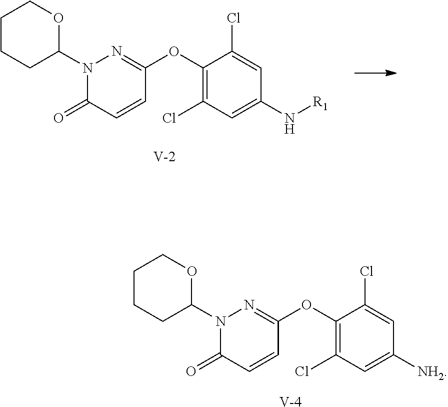

In the present disclosure, a preparation process of the compound having the structure shown in formula V-2 is the same as those described in the above solutions, and will not be repeated here.

In some embodiments of the present disclosure, the fifth alkaline compound is an alkali metal hydroxide. In some embodiments, the alkali metal hydroxide is potassium hydroxide and/or sodium hydroxide. In some embodiments, a molar ratio of the compound having the structure shown in formula V-2 to the fifth alkaline compound is in a range of 1:(2-6) and preferably 1:(2.5-4). In some embodiments, the fifth organic solvent adopted for the second hydrolysis reaction is at least one selected from the group consisting of ethanol, methanol, tetrahydrofuran, and DMSO, and preferably ethanol or methanol. In some embodiments, a volume ratio of the fifth organic solvent to the water is in a range of (1-4):1. In some embodiments, the second hydrolysis reaction is conducted at a temperature of 50° C. to 120° C. and preferably 60° C. to 100° C. In some embodiments, the second hydrolysis reaction is conducted for 1 h to 10 h and preferably 3 h to 8 h.

In some embodiments of the present disclosure, after the second hydrolysis reaction, a second hydrolysis reaction solution produced from the second hydrolysis reaction is subjected to a post-treatment. In some embodiments, the post-treatment includes the following steps: cooling the second hydrolysis reaction solution to room temperature, then adjusting with hydrochloric acid to a pH value of 6, subjecting a resulting mixture to rotary evaporation to remove a large amount of the fifth organic solvent and obtain a residue, and filtering the residue to obtain a solid product, and washing the solid product with water to obtain the compound having the structure shown in formula V (formula V-4).

In the present disclosure, under the condition that the compound having the structure shown in formula V is used to prepare the key intermediate III for Resmetirom, two preparation methods are provided according to whether R₁ and R₂ in formula V both are H in the present disclosure, which are described in detail below.

In the present disclosure, under the condition that in formula V, R₁ and R₂ both are H, the method for preparing the key intermediate III for Resmetirom includes the following step:
mixing the compound having the structure shown in formula V (namely, formula V-3), a compound having a structure shown in formula E, an alkaline compound (which is denoted as a sixth alkaline compound), and an organic solvent, and subjecting a resulting mixture to an addition-elimination-double bond migration reaction (which is denoted as a first addition-elimination-double bond migration reaction) to obtain the key intermediate III for Resmetirom, which has a structural formula shown in formula III:

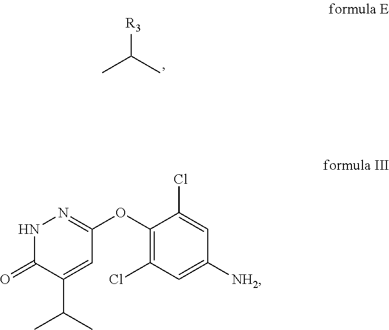

wherein in formula E, R₃ is selected from the group consisting of —NO₂, —SO₂Ph, —SO₂Me, —CN, —CF₃, and —CCl₃.

In some embodiments, under the condition that in formula V, R₁ and R₂ both are H, a synthesis route of the key intermediate III for Resmetirom is as follows:

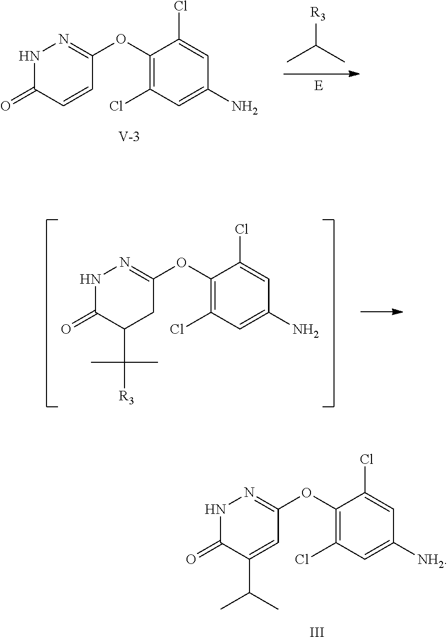

In some embodiments of the present disclosure, the sixth alkaline compound is at least one selected from the group consisting of DBU, 1,1,3,3-tetramethylguanidine, sodium amide, potassium tert-butoxide, sodium hydride, potassium carbonate, sodium carbonate, KOH, and NaOH, and preferably DBU or sodium amide. In some embodiments, a molar ratio of the compound having the structure shown in formula V to the sixth alkaline compound is in a range of 1:(0.5-10) and preferably 1:(1-5). In some embodiments, a molar ratio of the compound having the structure shown in formula V (namely, formula V-3) to the compound having the structure shown in formula E is in a range of 1:(1-5) and preferably 1:(1.1-3). In some embodiments, the organic solvent adopted for the addition-elimination-double bond migration reaction is one selected from the group consisting of DMSO, sulfolane, DMF, DMAc, NMP, tetrahydrofuran, and 1,4-dioxane, and preferably DMSO. In some embodiments, the first addition-elimination-double bond migration reaction is conducted at a temperature of 0° C. to 120° C. and preferably 20° C. to 80° C. In some embodiments, the first addition-elimination-double bond migration reaction is conducted for 1 h to 50 h and preferably 5 h to 30 h.

In some embodiments of the present disclosure, after the first addition-elimination-double bond migration reaction, a first addition-elimination-double bond migration reaction solution produced from the first addition-elimination-double bond migration reaction is subjected to a post-treatment. In some embodiments, the post-treatment includes the following steps: mixing the first addition-elimination-double bond migration reaction solution with water to obtain a mixed solution, adjusting the mixed solution with hydrochloric acid to a pH value of 6 and then filtering a resulting mixture to obtain a solid product, and subjecting the solid product to recrystallization to obtain the key intermediate III. In some embodiments, a solvent adopted for the recrystallization is a mixed solvent of acetonitrile and isopropyl ether. In some embodiments, a volume ratio of the acetonitrile to the isopropyl ether in the mixed solvent is in a range of (2-20):1.

In the present disclosure, under the condition that in formula V, R₁ and R₂ are not both H, the method for preparing the key intermediate III for Resmetirom includes the following steps:
(1) mixing the compound having the structure shown in formula V, a compound having the structure shown in formula E, an alkaline compound, and an organic solvent, and subjecting a resulting mixture to an addition-elimination-double bond migration reaction (which is denoted as a second addition-elimination-double bond migration reaction) to obtain a compound having a structure shown in formula IV,

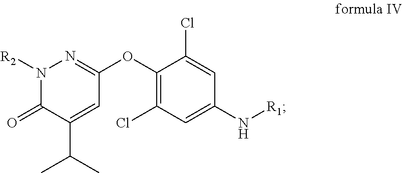

and
(2) removing R₁ and/or R₂ in the compound having the structure shown in formula IV through a deprotection reaction, to obtain the key intermediate III for Resmetirom.

In the present disclosure, specific conditions for the second addition-elimination-double bond migration reaction in step (1) are consistent with conditions for the first addition-elimination-double bond migration reaction in the above solutions. Operations for the second addition-elimination-double bond migration reaction are the same as operations for the first addition-elimination-double bond migration reaction, except that the compound having the structure shown in formula V-3 is correspondingly replaced with the compound having the structure shown in formula V where R₁ and R₂ are not both H. The type and amount of the alkaline compound are the same as those of the sixth alkaline compound, and will not be repeated here.

After the second addition-elimination-double bond migration reaction, the second addition-elimination-double bond migration reaction solution produced from the second addition-elimination-double bond migration reaction is subjected to the same post-treatment as the first addition-elimination-double bond migration reaction solution to obtain the compound having the structure shown in formula IV.

In the present disclosure, after the compound having the structure shown in formula IV is obtained, according to types of R₁ and R₂, an acidic deprotection reaction and/or an alkaline hydrolysis reaction are/is selected to remove the protecting group to obtain the key intermediate III for Resmetirom. A detailed description is given below.

In some embodiments of the present disclosure, under the condition that in formula IV, R₁ is t-BuOCO— and R₂ is H or

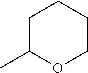

or under the condition that in formula IV, R₁ is H and R₂ is not H (which is denoted as situation 1), the deprotection reaction in step (2) is an acidic deprotection reaction. In some embodiments, an acid adopted for the acidic deprotection reaction is selected from the group consisting of a solution of hydrogen chloride in ethyl ether, a solution of hydrogen chloride in tetrahydrofuran, and a solution of hydrogen chloride in 1,4-dioxane. In some embodiments, in the solution of hydrogen chloride in ethyl ether, the solution of hydrogen chloride in tetrahydrofuran, or the solution of hydrogen chloride in 1,4-dioxane, a mass fraction of the hydrogen chloride is independently in a range of 10% to 50%. In some embodiments, a dosage ratio of the compound having the structure shown in formula IV to the acid is in a range of 1 g:(2-20) mL. In some embodiments, the acidic deprotection reaction is conducted at a temperature of 0° C. to 100° C. and preferably 20° C. to 80° C. In some embodiments, the acidic deprotection reaction is conducted for 1 h to 10 h.

In some embodiments of the present disclosure, after the acidic deprotection reaction, an acidic deprotection reaction solution produced from the acidic deprotection reaction is subjected to rotary evaporation until dryness to obtain a solid product, the solid product is washed with a sodium bicarbonate solution and then vacuum-dried to obtain a crude product, and the crude product is subjected to recrystallization to obtain the key intermediate III for Resmetirom. In some embodiments, a solvent adopted for the recrystallization is a mixed solvent of acetonitrile and isopropyl ether. In some embodiments, a volume ratio of the acetonitrile to the isopropyl ether in the mixed solvent is in a range of (2-20):1.

In some embodiments of the present disclosure, under the condition that in formula IV, R₁ is not H or t-BuOCO— and R₂ is H (which is denoted as situation 2), the deprotection reaction in step (2) is an alkaline hydrolysis reaction. In some embodiments, an alkali adopted for the alkaline hydrolysis reaction is sodium hydroxide or potassium hydroxide. In some embodiments, a molar ratio of the compound having the structure shown in formula IV to the alkali is in a range of 1:(2-10) and preferably 1:(3-8). In some embodiments, a solvent adopted for the alkaline hydrolysis reaction is an organic solvent-water mixed solvent. In some embodiments, an organic solvent in the organic solvent-water mixed solvent is at least one selected from the group consisting of ethanol, methanol, tetrahydrofuran, 1,4-dioxane, and DMSO, and preferably ethanol or methanol. In some embodiments, a volume ratio of the organic solvent to water in the organic solvent-water mixed solvent is in a range of (1-4):1. In some embodiments, the alkaline hydrolysis reaction is conducted at a temperature of 30° C. to 130° C. and preferably 50° C. to 110° C. In some embodiments, the alkaline hydrolysis reaction is conducted for 2 h to 20 h.

In some embodiments of the present disclosure, after the alkaline hydrolysis reaction, an alkaline hydrolysis reaction solution produced from the alkaline hydrolysis reaction is cooled to room temperature and then subjected to rotary evaporation to remove the organic solvent, ice water is added thereto, a pH value of a resulting mixture is adjusted with hydrochloric acid to 6 to precipitate a solid product; the solid product is separated through filtration, then washed with water, and dried to obtain a crude product; and the crude product is subjected to recrystallization to obtain the key intermediate III for Resmetirom. A solvent adopted for the recrystallization is consistent with that described in the above solutions, and will not be repeated here.

In the present disclosure, under the condition that in formula IV neither R₁ nor R₂ is H and R₁ is not t-BuOCO— (which is denoted as situation 3), the deprotection reaction in step (2) includes an acidic deprotection reaction and an alkaline hydrolysis reaction that are conducted sequentially. Conditions for the acidic deprotection reaction in situation 3 are the consistent with the conditions in situation 1, and will not be repeated here. Conditions for the alkaline hydrolysis reaction in situation 3 are consistent with conditions in situation 2, and will not be repeated here. In specific embodiments of the present disclosure, after the acidic deprotection reaction, an acidic deprotection reaction solution is subjected to rotary evaporation until dryness, and then a reagent for alkaline hydrolysis is added thereto and the alkaline hydrolysis reaction occurs. A post-treatment after the alkaline hydrolysis reaction is consistent with the post-treatment in situation 2, and will not be repeated here.

The technical solutions in the present disclosure are clearly and completely described below with reference to the embodiments of the present disclosure. Apparently, the described embodiments are merely some rather than all of the embodiments of the present disclosure. All other embodiments obtained by a person of ordinary skill in the art based on the embodiments of the present disclosure without creative efforts shall fall within the scope of the present disclosure.

### Example 1 Preparation of a Compound Having a Structural Formula V-1a

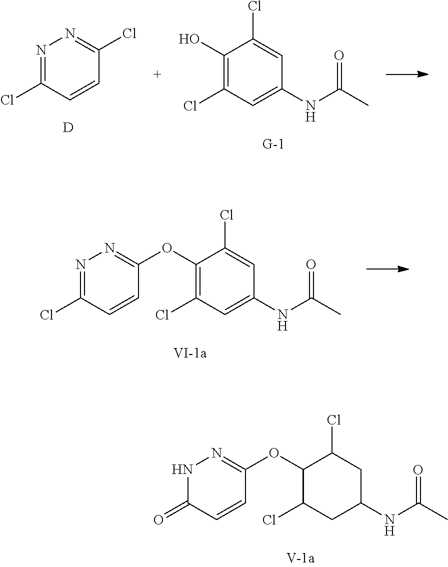

Method 1: 14.9 g of a compound D (100 mmol), 26.4 g of a compound having a structural formula G-1 (120 mmol), 20.7 g of a potassium carbonate powder (150 mmol), and 200 mL of DMF were added to a reaction flask, and a resulting mixture was heated to 65° C. and reacted at 65° C. with stirring for 24 h. The resulting reaction mixture was poured into 1,000 mL of water. The resulting system was adjusted with liquid caustic soda to a pH value of 11, stirred at room temperature for 30 min, and filtered to obtain a filter cake. The filter cake was washed with 100 mL of water and then oven-dried to obtain a compound VI-1a. 120 mL of glacial acetic acid and 16.4 g of sodium acetate (200 mmol) were added to the compound VI-1a, and the resulting mixture was heated to 110° C. and reacted at 110° C. for 5 h. The resulting reaction system was subjected to rotary evaporation to remove the glacial acetic acid, and 150 mL of water was added thereto. The resulting mixture was stirred at room temperature for 2 h and then filtered to obtain a filter cake. The filter cake was washed with 50 mL of water and oven-dried to obtain 29.2 g of an off-white solid, i.e., compound V-1a, with a yield of 93%.

Method 2: 14.9 g of a compound D (100 mmol), 24.2 g of a compound having a structural formula G-1 (110 mmol), 21.2 g of a sodium carbonate powder (200 mmol), and 200 mL of DMSO were added to a reaction flask, and a resulting mixture was heated to 75° C. and reacted at 75° C. while stirring for 16 h. The resulting reaction mixture was poured into 1,000 mL of water. The resulting system was adjusted with liquid caustic soda to a pH value of 11, stirred at room temperature for 30 min, and filtered to obtain a filter cake. The resulting filter cake was washed with 100 ml of water and then oven-dried to obtain a compound VI-1a. 100 mL of glacial acetic acid and 12.3 g of sodium acetate (150 mmol) were added to the compound VI-1a. The resulting system was heated to 100° C. and reacted at 100° C. for 8 h, and the resulting reaction mixture was subjected to rotary evaporation to remove the glacial acetic acid, and 150 mL of water was added thereto. The resulting system was stirred at room temperature for 2 h and filtered to obtain a filter cake. The filter cake was washed with 50 ml of water and then oven-dried to obtain 28.3 g of an off-white solid, i.e., compound V-1a, with a yield of 90%.

Nuclear magnetic resonance data for the compound V-1a obtained by Method 1 was as follows: ¹H-NMR (400M, DMSO-d₆): 12.27 (1H, s), 10.30 (1H, s), 7.77 (2H, s), 7.58 (1H, d), 7.07 (1H, d), 2.07 (3H, s).

### Example 2 Preparation of a Compound Having a Structural Formula V-1b

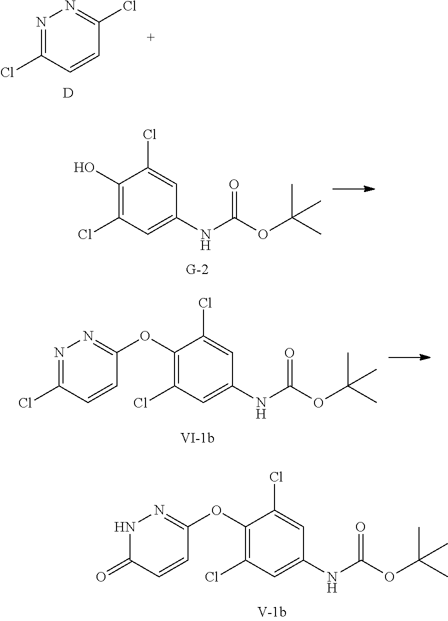

14.9 g of a compound D (100 mmol), 30.6 g of a compound having a structural formula G-2 (110 mmol), 42.4 g of a cesium carbonate powder (130 mmol), and 200 mL of DMAc were added to a reaction flask, and a resulting mixture was heated to 65° C. and reacted at 65° C. with stirring for 24 h. The resulting reaction mixture was poured into 1,000 mL of water. The resulting system was adjusted with liquid caustic soda to a pH value of 11, stirred at room temperature for 30 min, and filtered to obtain a filter cake. The resulting filter cake was washed with 100 mL of water and then oven-dried to obtain a compound VI-1b. 120 mL of glacial acetic acid and 16.4 g of sodium acetate (200 mmol) were added to the compound VI-1b. The resulting system was heated to 110° C. and reacted at 110° C. for 5 h. The resulting reaction mixture was subjected to rotary evaporation to remove the glacial acetic acid, and 150 ml of water was then added thereto. The resulting system was stirred at room temperature for 2 h and then filtered to obtain a filter cake. The filter cake was washed with 50 mL of water and oven-dried to obtain 30.9 g of an off-white solid, i.e., compound V-1b, with a yield of 83%, ¹H-NMR (400M, DMSO-d₆): 12.23 (1H, s), 10.09 (1H, s), 7.75 (2H, s), 7.57 (1H, d), 7.01 (1H, d), 1.28 (9H, s).

### Example 3 Preparation of a Compound Having a Structural Formula V-1c

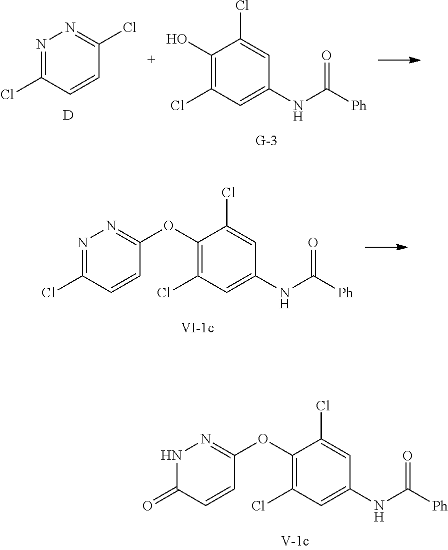

14.9 g of a compound D (100 mmol), 31.0 g of a compound having a structural formula G-3 (110 mmol), 15.7 g of a potassium tert-butoxide powder (140 mmol), and 200 mL of DMF were added to a reaction flask, and a resulting mixture was heated to 65° C. and reacted at 65° C. with stirring for 24 h. The resulting reaction mixture was poured into 1,000 mL of water. The resulting system was adjusted with liquid caustic soda to a pH value of 11, stirred at room temperature for 30 min, and filtered to obtain a filter cake. The resulting filter cake was washed with 100 ml of water and then oven-dried to obtain a compound VI-1c. 120 mL of glacial acetic acid and 13.1 g of sodium acetate (160 mmol) were added to the compound VI-1c. The resulting system was heated to 105° C. and reacted at 105° C. for 8 h, and the resulting reaction mixture was subjected to rotary evaporation to remove the glacial acetic acid, and 150 ml of water was added thereto. The resulting mixture was stirred at room temperature for 2 h and then filtered to obtain a filter cake. The filter cake was washed once with 50 mL of water and oven-dried to obtain 32.7 g of an off-white solid, i.e., compound V-1c, with a yield of 87%, ¹H-NMR (400M, DMSO-d₆): 12.25 (1H, s), 10.26 (1H, s), 7.98 (2H, m), 7.76 (2H, s), 7.63 (1H, m), 7.58 (1H, d), 7.47 (2H, m), 7.05 (1H, d).

### Example 4 Preparation of a Compound Having a Structural Formula V-1d

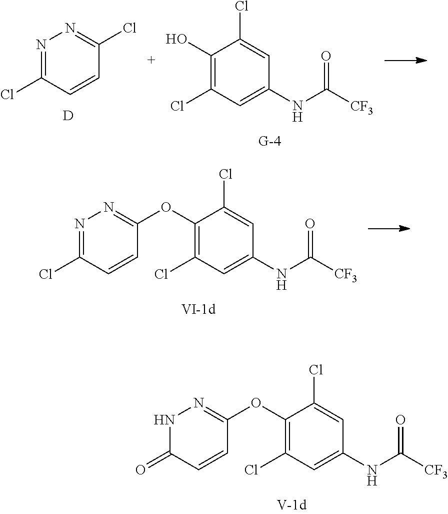

14.9 g of a compound D (100 mmol), 31.0 g of a compound having a structural formula G-4 (110 mmol), 20.7 g of a potassium carbonate powder (150 mmol), and 200 mL of DMF were added to a reaction flask, and the resulting mixture was heated to 65° C. and reacted at 65° C. with stirring for 24 h. The resulting reaction mixture was poured into 1,000 mL of water. The resulting system was adjusted with liquid caustic soda to a pH value of 11, stirred at room temperature for 30 min, and filtered to obtain a filter cake. The resulting filter cake was washed with 100 mL of water and then oven-dried to obtain a compound VI-1d. 120 mL of glacial acetic acid and 13.1 g of sodium acetate (160 mmol) were added to the compound VI-1d. The resulting system was heated to 105° C. and reacted at 105° C. for 8 h. The resulting reaction mixture was subjected to rotary evaporation to remove the glacial acetic acid, and 150 mL of water was added thereto. The resulting system was stirred at room temperature for 2 h and then filtered to obtain a filter cake. The filter cake was washed with 50 mL of water and oven-dried to obtain 33.9 g of an off-white solid, i.e., compound V-1d, with a yield of 92%, ¹H-NMR (400M, DMSO-d₆): 12.27 (1H, s), 10.54 (1H, s), 7.85 (2H, s), 7.58 (1H, d), 7.05 (1H, d).

### Example 5 Preparation of a Compound Having a Structural Formula V-1e

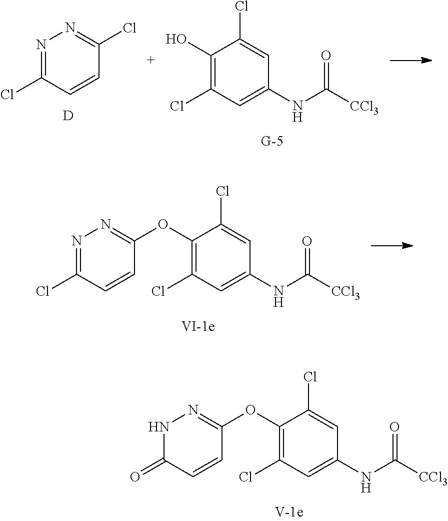

14.9 g of a compound D (100 mmol), 34.0 g of a compound G-5 (105 mmol), 22.1 g of a potassium carbonate powder (160 mmol), and 200 mL of DMAc were added to a reaction flask, and a resulting mixture was heated to 80° C. and reacted with stirring at 80° C. for 12 h. The resulting reaction mixture was poured into 1,000 mL of water. The resulting system was adjusted with liquid caustic soda to a pH value of 11, stirred at room temperature for 30 min, and filtered to obtain a filter cake. The resulting filter cake was washed with 100 ml of water to obtain a compound VI-1e. 10.7 g of sodium acetate (130 mmol) and 80 mL of glacial acetic acid were added to the compound VI-1e. The resulting system was heated to 100° C. and reacted at 100° C. for 8 h. The resulting reaction mixture was subjected to rotary evaporation to remove the glacial acetic acid, and 100 mL of water was added thereto. The resulting system was stirred for 10 min and then filtered to obtain a filter cake. The filter cake was washed with 50 ml of water and oven-dried to obtain 38.4 g of an off-white solid, i.e., compound V-1e, with a yield of 92%. ¹H-NMR (400M, DMSO-d₆): 12.26 (1H, s), 10.45 (1H, s), 7.83 (2H, s), 7.58 (1H, d), 7.05 (1H, d).

### Example 6 Preparation of a Compound Having a Structural Formula V-2a

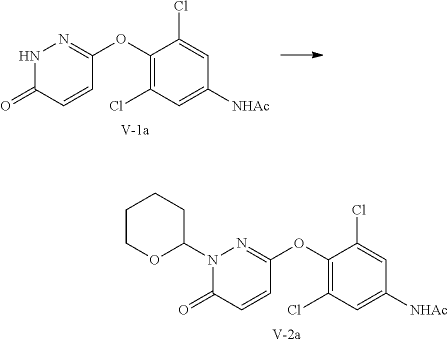 [SMILES: CC(=O)Nc1cc(Cl)c(Oc2ccc(=O)[nH]n2)c(Cl)c1 ; CC(=O)Nc1cc(Cl)c(Oc2ccc(=O)n(C3CCCCO3)n2)c(Cl)c1]

Method 1: 9.42 g of a compound V-1a (30 mmol), 3.78 g of 3,4-dihydro-2H-pyran (45 mmol), 0.52 g of p-toluenesulfonic acid (3.0 mmol), and 100 mL of tetrahydrofuran were added to a reaction flask, and a resulting mixture was heated to 60° C. and reacted at 60° C. for 5 h. The resulting reaction mixture was cooled to room temperature, and 100 ml of water was added thereto. The resulting system was adjusted with potassium carbonate to a pH value of 9, stirred at room temperature for 30 min, and subjected to rotary evaporation to remove tetrahydrofuran and precipitate a large amount of a solid. The solid was separated through filtration, washed with 50 mL of a solvent consisting of ethyl acetate and heptane in a ratio of 1:2, and oven-dried to obtain 11.8 g of an off-white solid, i.e., compound V-2a, with a yield of 99%.

Method 2: 9.42 g of a compound V-1a (30 mmol), 5.04 g of 3,4-dihydro-2H-pyran (60 mmol), 0.77 g of methanesulfonic acid (8.0 mmol), and 100 mL of acetonitrile were added to a reaction flask, and the resulting mixture was heated to 70° C. and reacted at 70° C. for 3 h. The resulting reaction mixture was cooled to room temperature, and 100 ml of water was added thereto. The resulting system was adjusted with potassium carbonate to a pH value of 9, stirred at room temperature for 30 min, and subjected to rotary evaporation to remove acetonitrile and precipitate a large amount of a solid. The solid was separated through filtration, washed with 50 mL of a solvent consisting of ethyl acetate and heptane in a ratio of 1:2, and oven-dried to obtain 11.6 g of an off-white solid, i.e., compound V-2a, with a yield of 97%.

Nuclear magnetic resonance data for the compound V-2a obtained by Method 1 was as follows: ¹H-NMR (400M, DMSO-d₆): 10.31 (1H, brs), 7.80 (2H, s), 7.60 (1H, d), 7.17 (1H, d), 5.69 (1H, dd), 3.79 (1H, d), 3.49 (1H, t), 2.08 (3H, s), 1.49-1.22 (6H, m).

### Example 7 Preparation of a Compound Having a Structural Formula V-2b

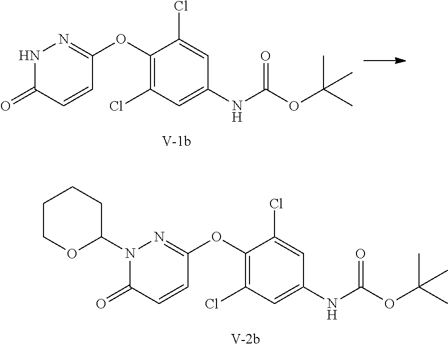 [SMILES: CC(C)(C)OC(=O)Nc1cc(Cl)c(Oc2ccc(=O)[nH]n2)c(Cl)c1 ; CC(C)(C)OC(=O)Nc1cc(Cl)c(Oc2ccc(=O)n(C3CCCCO3)n2)c(Cl)c1]

11.17 g of a compound V-1b (30 mmol), 3.78 g of 3,4-dihydro-2H-pyran (45 mmol), 0.46 g of trifluoroacetic acid (4.0 mmol), and 100 mL of ethyl acetate were added to a reaction flask, and the resulting mixture was heated to 60° C. and reacted at 60° C. for 5 h. The resulting reaction mixture was cooled to room temperature, and 100 ml of water was added thereto. The resulting system was adjusted with potassium carbonate to a pH value of 9, stirred at room temperature for 30 min, and subjected to rotary evaporation to remove ethyl acetate and precipitate a large amount of a solid. The solid was separated through filtration, washed with 50 mL of a solvent consisting of ethyl acetate and heptane in a ratio of 1:2, and oven-dried to obtain 11.6 g of an off-white solid, i.e., compound V-2b, with a yield of 85%.

¹H-NMR (400M, DMSO-d₆): 10.09 (1h, brs), 7.71 (2H, s), 7.55 (1H, d), 7.13 (1H, d), 5.65 (1H, dd), 3.74 (1H, m), 3.53 (1H, m), 1.58-1.26 (15H, m).

### Example 8 Preparation of a Compound Having a Structural Formula V-2c

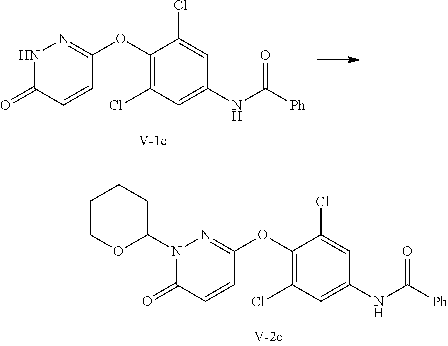 [SMILES: O=C(Nc1cc(Cl)c(Oc2ccc(=O)[nH]n2)c(Cl)c1)c1ccccc1 ; O=C(Nc1cc(Cl)c(Oc2ccc(=O)n(C3CCCCO3)n2)c(Cl)c1)c1ccccc1]

11.3 g of a compound V-1c (30 mmol), 5.04 g of 3,4-dihydro-2H-pyran (60 mmol), 1.04 g of p-toluenesulfonic acid (6.0 mmol), and 100 mL of tetrahydrofuran were added to a reaction flask, and the resulting mixture was heated to 60° C. and reacted at 60° C. for 5 h. The resulting reaction mixture was cooled to room temperature, and 100 ml of water was added thereto. The resulting system was adjusted with potassium carbonate to a pH value of 9, stirred at room temperature for 30 min, and subjected to rotary evaporation to remove tetrahydrofuran and precipitate a large amount of a solid. The solid was separated through filtration, washed with 50 mL of a solvent consisting of ethyl acetate and heptane in a ratio of 1:2, and oven-dried to obtain 13.5 g of an off-white solid, i.e., compound V-2c, with a yield of 98%, ¹H-NMR (400M, DMSO-d₆): 10.21 (1h, brs), 7.98 (2H, m), 7.80 (2H, s), 7.63 (1H, m), 7.56-7.47 (3H, m), 7.15 (1H, d), 5.65 (1H, dd), 3.76 (1H, m), 3.53 (1H, m), 1.59-1.28 (6H, m).

### Example 9 Preparation of a Compound Having a Structural Formula V-2d

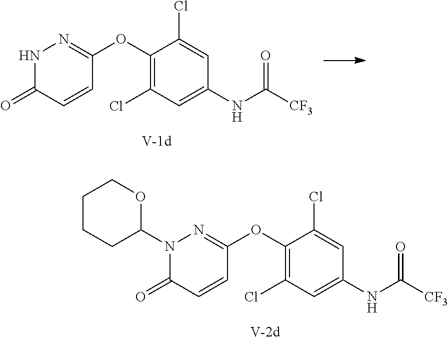 [SMILES: O=C(Nc1cc(Cl)c(Oc2ccc(=O)[nH]n2)c(Cl)c1)C(F)(F)F ; O=C(Nc1cc(Cl)c(Oc2ccc(=O)n(C3CCCCO3)n2)c(Cl)c1)C(F)(F)F]

11.0 g of a compound V-1d (30 mmol), 5.04 g of 3,4-dihydro-2H-pyran (60 mmol), 1.39 g of p-toluenesulfonic acid (8.0 mmol), and 100 mL of acetone were added to a reaction flask, and the resulting mixture was heated to 60° C. and reacted at 60° C. for 5 h. The resulting reaction mixture was cooled to room temperature, and 100 mL of water was added thereto. The resulting system was adjusted with potassium carbonate to a pH value of 9, stirred at room temperature for 30 min, and subjected to rotary evaporation to remove acetone and precipitate a large amount of a solid. The solid was separated through filtration, washed with 50 mL of a solvent consisting of ethyl acetate and heptane in a ratio of 1:2, and oven-dried to obtain 13.0 g of an off-white solid, i.e., compound V-2d, with a yield of 96%, ¹H-NMR (400M, DMSO-d₆): 10.52 (1h, brs), 7.85 (2H, s), 7.61 (1H, d), 7.19 (1H, d), 5.66 (1H, dd), 3.79 (1H, m), 3.54 (1H, m), 1.59-1.28 (6H, m).

### Example 10 Preparation of a Compound Having a Structural Formula V-2e

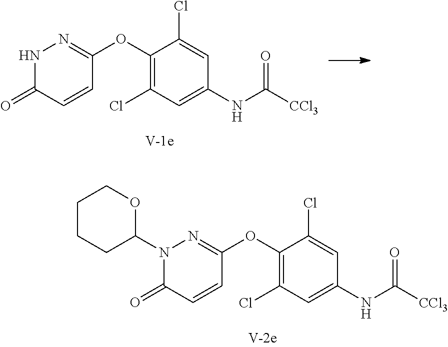

12.5 g of a compound V-1e (30 mmol), 5.04 g of 3,4-dihydro-2H-pyran (60 mmol), 2.08 g of p-toluenesulfonic acid (12.0 mmol), and 100 mL of tetrahydrofuran were added to a reaction flask, and the resulting mixture was heated to 60° C. and reacted at 60° C. for 5 h. The resulting reaction mixture was cooled to room temperature, and 100 ml of water was added thereto. The resulting system was adjusted with potassium carbonate to a pH value of 9, stirred at room temperature for 30 min, and subjected to rotary evaporation to remove tetrahydrofuran and precipitate a large amount of a solid. The solid was separated through filtration, washed with 50 mL of a solvent consisting of ethyl acetate and heptane in a ratio of 1:2, and oven-dried to obtain 14.6 g of an off-white solid, i.e., compound V-2e, with a yield of 97%, ¹H-NMR (400M, DMSO-d₆): 10.46 (1h, brs), 7.84 (2H, s), 7.61 (1H, d), 7.19 (1H, d), 5.65 (1H, dd), 3.78 (1H, m), 3.52 (1H, m), 1.59-1.28 (6H, m).

### Example 11 Preparation of a Compound Having a Structural Formula V-3

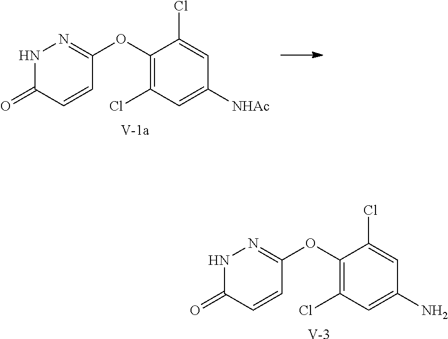

Method 1: 9.42 g of the compound V-1a (30 mmol), 100 mL of ethanol, 50 mL of water, and 5.6 g of potassium hydroxide (100 mmol) were added to a reaction flask, and the resulting mixture was heated to 90° C. and reacted at 90° C. for 5 h. The resulting reaction mixture was cooled to room temperature, adjusted with hydrochloric acid to a pH value of 6, and subjected to rotary evaporation to remove ethanol and precipitate a large amount of a solid. The solid was separated through filtration, washed with 50 mL of water, and oven-dried to obtain 7.83 g of a brown-yellow solid, i.e., compound V-3, with a yield of 96%.

Method 2: 9.42 g of the compound V-1a (30 mmol), 100 mL of tetrahydrofuran, 50 mL of water, and 4.8 g of sodium hydroxide (120 mmol) were added to a reaction flask, and the resulting mixture was heated to 80° C. and reacted at 80° C. for 10 h. The resulting reaction mixture was cooled to room temperature, adjusted with hydrochloric acid to a pH value of 6, and subjected to rotary evaporation to remove tetrahydrofuran and precipitate a large amount of a solid. The solid was separated through filtration, washed with 50 mL of water, and oven-dried to obtain 7.75 g of a brown-yellow solid, i.e., compound V-3, with a yield of 95%.

Nuclear magnetic resonance data for the compound V-3 obtained by Method 1 was as follows: ¹H-NMR (400M, DMSO-d₆): 12.22 (1h, brs), 7.50 (1H, d), 7.03 (1H, d), 6.68 (2H, s), 5.63 (2H, brs).

### Example 12 Preparation of a Compound Having a Structural Formula V-3

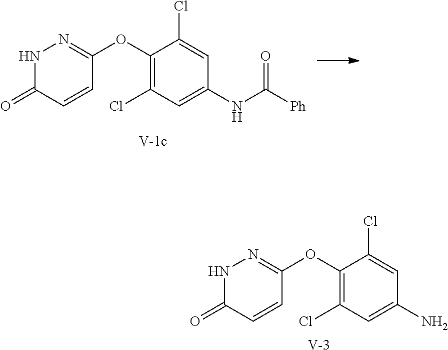

11.3 g of a compound V-1c (30 mmol), 100 mL of ethanol, 50 mL of water, and 6.0 g of sodium hydroxide (150 mmol) were added to a reaction flask, and the resulting mixture was heated to 100° C. and reacted at 100° C. for 5 h. The resulting reaction mixture was cooled to room temperature, adjusted with hydrochloric acid to a pH value of 6, and subjected to rotary evaporation to remove ethanol and precipitate a large amount of a solid. The solid was separated through filtration, washed with 50 mL of water, and oven-dried to obtain 8.0 g of a brown-yellow solid, i.e., compound V-3, with a yield of 98%. The nuclear magnetic resonance data in this example was consistent with the nuclear magnetic resonance data in Example 11.

### Example 13 Preparation of a Compound Having a Structural Formula V-3

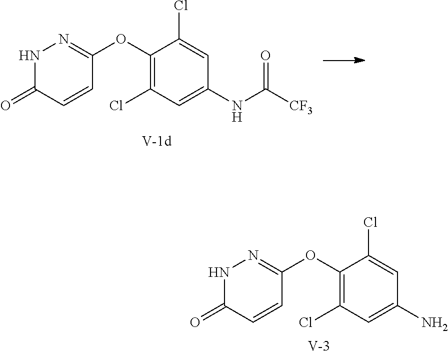

11.0 g of a compound V-1d (30 mmol), 100 mL of ethanol, 50 mL of water, and 5.6 g of potassium hydroxide (100 mmol) were added to a reaction flask, and the resulting mixture was heated to 80° C. and reacted at 80° C. for 2 h. The resulting reaction mixture was cooled to room temperature, adjusted with hydrochloric acid to a pH value of 6, and subjected to rotary evaporation to remove ethanol and precipitate a large amount of a solid. The solid was separated through filtration, washed with 50 mL of water, and oven-dried to obtain 8.0 g of a brown-yellow solid, i.e., compound V-3, with a yield of 98%. The nuclear magnetic resonance data in this example was consistent with the nuclear magnetic resonance data in Example 11.

### Example 14 Preparation of a Compound Having a Structural Formula V-3

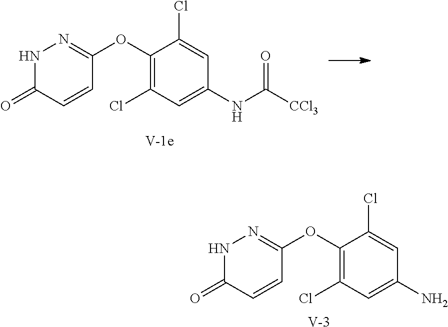

12.5 g of a compound V-1e (30 mmol), 100 mL of tetrahydrofuran, 50 mL of water, and 8.0 g of sodium hydroxide (200 mmol) were added to a reaction flask, and the resulting mixture was heated to 80° C. and reacted at 80° C. for 2 h. The resulting reaction mixture was cooled to room temperature, adjusted with hydrochloric acid to a pH value of 6, and subjected to rotary evaporation to remove tetrahydrofuran and precipitate a large amount of a solid. The solid was separated through filtration, washed with 50 mL of water, and oven-dried to obtain 8.0 g of a brown-yellow solid, i.e., compound V-3, with a yield of 98%. The nuclear magnetic resonance data in this example was consistent with the nuclear magnetic resonance data in Example 11.

### Example 15 Preparation of a Compound Having a Structural Formula V-4

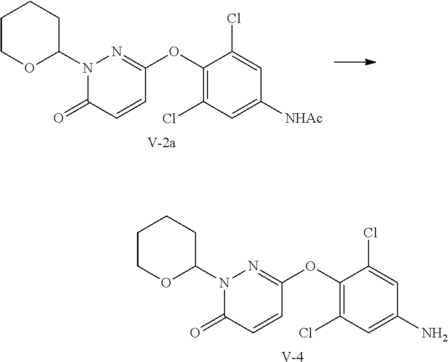

12.0 g of the compound V-2a (30 mmol), 100 mL of ethanol, 50 mL of water, and 3.36 g of potassium hydroxide (60 mmol) were added to a reaction flask, and the resulting mixture was heated to 90° C. and reacted at 90° C. for 6 h. The resulting reaction mixture was cooled to room temperature, adjusted with hydrochloric acid to a pH value of 6, and subjected to rotary evaporation to remove ethanol and precipitate a large amount of a solid. The solid was separated through filtration, washed with 50 mL of water, and oven-dried to obtain 8.76 g of a pale-yellow solid, i.e., compound V-4, with a yield of 82%, ¹H-NMR (400M, DMSO-d₆): 7.56 (1H, d), 7.15 (1H, d), 6.68 (2H, s), 5.76 (2H, brs), 5.63 (1H, dd), 3.78 (1H, m), 3.52 (1H, m), 1.51-1.25 (6H, m).

### Example 16 Preparation of a Compound Having a Structural Formula V-4

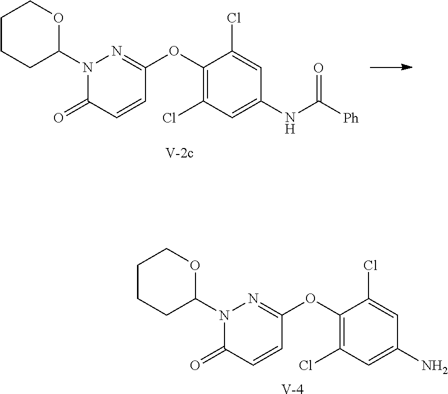

13.8 g of the compound V-2c (30 mmol), 100 mL of methanol, 50 ml of water, and 4.8 g of sodium hydroxide (120 mmol) were added to a reaction flask, and the resulting mixture was heated to 100° C. and reacted at 100° C. for 5 h. The resulting reaction mixture was cooled to room temperature, adjusted with hydrochloric acid to a pH value of 6, and subjected to rotary evaporation to remove methanol and precipitate a large amount of a solid. The solid was separated through filtration, washed with 50 mL of water, and oven-dried to obtain 9.6 g of a pale-yellow solid, i.e., compound V-4, with a yield of 90%. The nuclear magnetic resonance data in this example was consistent with the nuclear magnetic resonance data in Example 15.

### Example 17 Preparation of a Compound Having a Structural Formula V-4

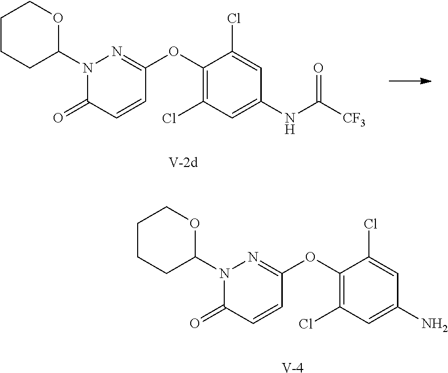

13.6 g of the compound V-2d (30 mmol), 100 mL of ethanol, 50 mL of water, and 5.04 g of potassium hydroxide (90 mmol) were added to a reaction flask, and the resulting mixture was heated to 70° C. and reacted at 70° C. for 3 h. The resulting reaction mixture was cooled to room temperature, adjusted with hydrochloric acid to a pH value of 6, and subjected to rotary evaporation to remove ethanol and precipitate a large amount of a solid. The solid was separated through filtration, washed with 50 mL of water, and oven-dried to obtain 9.7 g of a pale-yellow solid, i.e., compound V-4, with a yield of 91%. The nuclear magnetic resonance data in this example was consistent with the nuclear magnetic resonance data in Example 15.

### Example 18 Preparation of a Compound Having a Structural Formula V-4

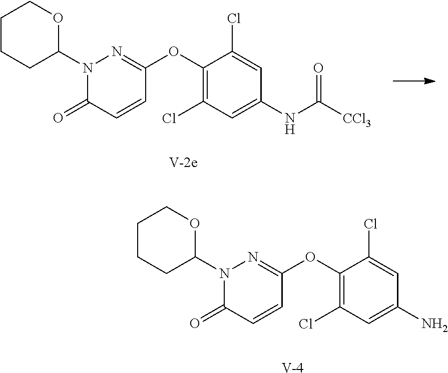

15.0 g of the compound V-2e (30 mmol), 100 mL of methanol, 50 mL of water, and 4.0 g of sodium hydroxide (100 mmol) were added to a reaction flask, and the resulting mixture was heated to 70° C. and reacted at 70° C. for 3 h. The resulting reaction mixture was cooled to room temperature, adjusted with hydrochloric acid to a pH value of 6, and subjected to rotary evaporation to remove methanol and precipitate a large amount of a solid. The solid was separated through filtration, washed with 50 mL of water, and oven-dried to obtain 9.6 g of a pale-yellow solid, i.e., compound V-4, with a yield of 90%. The nuclear magnetic resonance data in this example was consistent with the nuclear magnetic resonance data in Example 15.

### Example 19 Preparation of a Compound Having a Structural Formula III

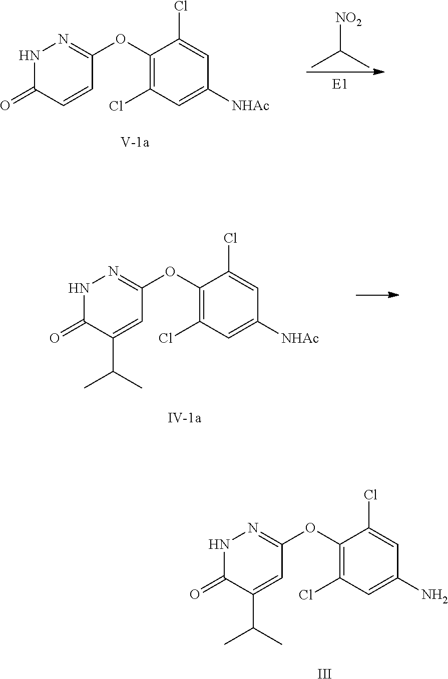

3.14 g of the compound V-1a (10 mmol), 1.34 g of a compound E-1 (15 mmol), 3.45 g of 1,1,3,3-tetramethylguanidine (30 mmol), and 30 mL of DMSO were added to a reaction flask, and the resulting mixture was stirred at 50° C. and reacted at 50° C. for 20 h. The resulting reaction mixture was poured into 120 mL of ice water. The resulting system was adjusted with hydrochloric acid to a pH value of 6 and filtered to obtain a filter cake. The filter cake was washed once with 20 mL of water to obtain a compound IV-1a. 20 mL of ethanol, 10 mL of water, and 2.0 g of sodium hydroxide (50 mmol) were added to the compound IV-1a, and the resulting system was heated to 100° C. and reacted at 100° C. for 5 h. The resulting reaction mixture was cooled to room temperature, and subjected to rotary evaporation to remove ethanol. 20 mL of ice water was then added thereto, and the resulting system was adjusted with hydrochloric acid to a pH value of 6 such that a large amount of a solid was precipitated. The solid was separated through filtration, washed once with 20 mL of water, and then vacuum-dried to obtain a crude product. The crude product was subjected to recrystallization with acetonitrile and isopropyl ether to obtain 2.54 g of an off-white solid, i.e., compound III, with a yield of 81%. It was identified by nuclear magnetic resonance that the product had a target structure.

### Example 20 Preparation of a Compound Having a Structural Formula III

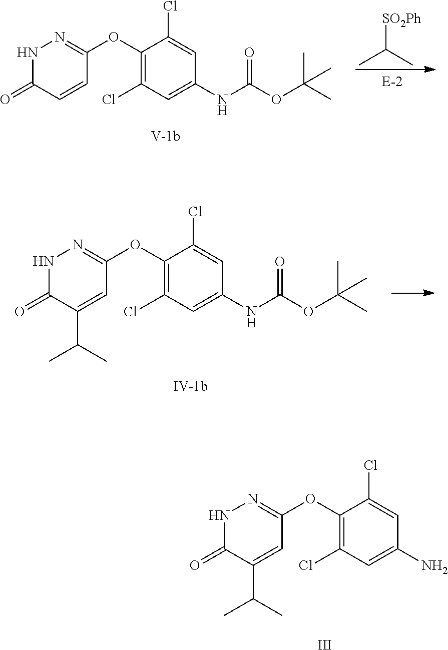

3.72 g of a compound V-1b (10 mmol), 2.2 g of a compound E-2 (12 mmol), 1.29 g of sodium amide (33 mmol), and 30 mL of DMAc were added to a reaction flask, and the resulting mixture was subjected to a reaction at 40° C. for 15 h. The resulting reaction mixture was poured into 120 mL of ice water. The resulting system was adjusted with hydrochloric acid to a pH value of 6 and filtered to obtain a filter cake. The filter cake was washed once with 20 ml of water and vacuum-dried to obtain a compound IV-1b. 10 mL of a solution of hydrogen chloride in ethyl ether was added to the compound IV-1b, and the resulting mixture was stirred at room temperature and reacted for 2 h. The resulting reaction mixture was subjected to rotary evaporation until dryness, then washed with 20 mL of a sodium bicarbonate solution, and vacuum-dried to obtain a crude product. The crude product was subjected to recrystallization with acetonitrile and isopropyl ether to obtain 2.61 g of an off-white solid, i.e., compound III, with a yield of 83%. Nuclear magnetic resonance data for the product was as follows: ¹H-NMR (400M, DMSO-d₆): 12.12 (1H, s), 7.27 (1H, s), 6.67 (2H, s), 5.61 (2H, brs), 3.03 (1H, m), 1.17 (6H, d). A nuclear magnetic resonance spectrum is shown in FIGURE.

### Example 21 Preparation of a Compound Having a Structural Formula III

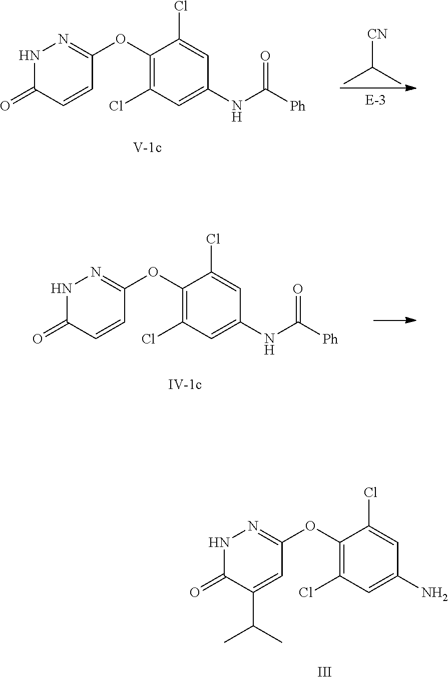

3.76 g of a compound V-1c (10 mmol), 1.38 g of a compound E-3 (20 mmol), 1.08 g of sodium hydride (45 mmol), and 40 mL of DMF were added to a reaction flask, and the resulting mixture was stirred at 60° C. and reacted at 60° C. for 12 h. 150 ml of water and 1.5 g of sodium hypochlorite were added to the reaction flask, and the resulting system was stirred at room temperature for 5 h and filtered to obtain a filter cake. The filter cake was washed once with 20 mL of water to obtain a compound IV-1c. 20 mL of methanol, 10 mL of water, and 2.8 g of potassium hydroxide (50 mmol) were added to the compound IV-1c, and the resulting mixture was heated to 90° C. and reacted at 90° C. for 8 h. The resulting reaction mixture was cooled to room temperature, and subjected to rotary evaporation to remove methanol. 20 mL of ice water was added thereto, and the resulting system was adjusted with hydrochloric acid to a pH value of 6 such that a large amount of a solid was precipitated. The solid was separated through filtration, washed once with 20 mL of water, and then vacuum-dried to obtain a crude product. The crude product was subjected to recrystallization with acetonitrile and isopropyl ether to obtain 2.51 g of an off-white solid, i.e., compound III, with a yield of 80%. It was identified by nuclear magnetic resonance that the product had a target structure.

### Example 22 Preparation of a Compound Having a Structural Formula III

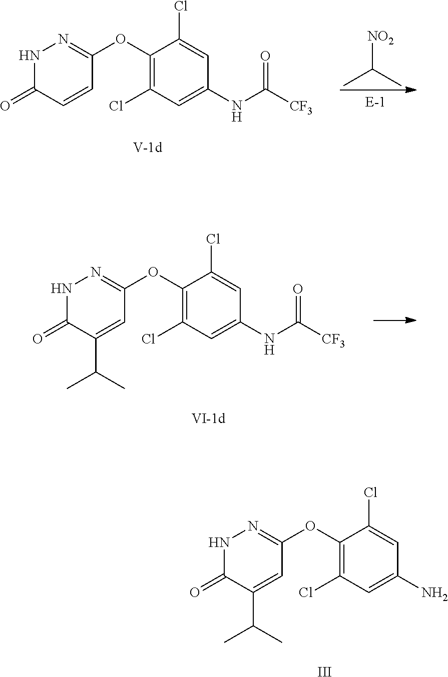

3.68 g of the compound V-1d (10 mmol), 1.07 g of a compound E-1 (12 mmol), 1.83 g of DBU (12 mmol), and 30 mL of sulfolane were added to a reaction flask, and the resulting mixture was reacted at 50° C. with stirring for 20 h. The resulting reaction mixture was poured into 120 mL of ice water. The resulting system was adjusted with hydrochloric acid to a pH value of 6 and filtered to obtain a filter cake. The filter cake was washed once with 20 ml of water to obtain a compound VI-1d. 20 mL of ethanol, 10 mL of water, and 2.0 g of sodium hydroxide (50 mmol) were added to the compound VI-1d. The resulting mixture was heated to 80° C. and reacted at 80° C. for 2 h. The resulting reaction mixture was cooled to room temperature, and subjected to rotary evaporation to remove ethanol. 20 mL of ice water was added thereto, and the resulting system was adjusted with hydrochloric acid to a pH value of 6 such that a large amount of a solid was precipitated. The solid was separated through filtration, washed once with 20 mL of water, and then vacuum-dried to obtain a crude product. The crude product was subjected to recrystallization with acetonitrile and isopropyl ether to obtain 2.70 g of an off-white solid, i.e., compound III, with a yield of 85%. It was identified by nuclear magnetic resonance that the product had a target structure.

### Example 23 Preparation of a Compound Having a Structural Formula III

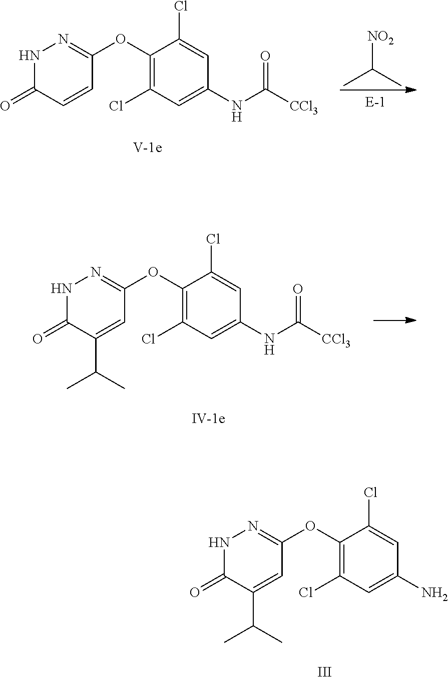

4.18 g of the compound V-1e (10 mmol), 1.07 g of a compound E-1 (12 mmol), 1.83 g of DBU (12 mmol), and 30 mL of DMSO were added to a reaction flask, and the resulting mixture was reacted at 50° C. with stirring for 20 h. The resulting reaction mixture was poured into 120 mL of ice water. The resulting system was adjusted with hydrochloric acid to a pH value of 6 and filtered to obtain a filter cake. The filter cake was washed once with 20 ml of water to obtain a compound VI-1e. 20 mL of ethanol, 10 mL of water, and 2.0 g of sodium hydroxide (50 mmol) were added to the compound VI-1e, and the resulting system was heated to 80° C. and reacted at 80° C. for 2 h. The resulting reaction mixture was cooled to room temperature, and subjected to rotary evaporation to remove ethanol. 20 mL of ice water was added thereto, and the resulting system was adjusted with hydrochloric acid to a pH value of 6 such that a large amount of a solid was precipitated. The solid was separated through filtration, washed once with 20 mL of water, and then vacuum-dried to obtain a crude product. The crude product was subjected to recrystallization with acetonitrile and isopropyl ether to obtain 2.64 g of an off-white solid, i.e., compound III, with a yield of 84%. It was identified by nuclear magnetic resonance that the product had a target structure.

### Example 24 Preparation of a Compound Having a Structural Formula III

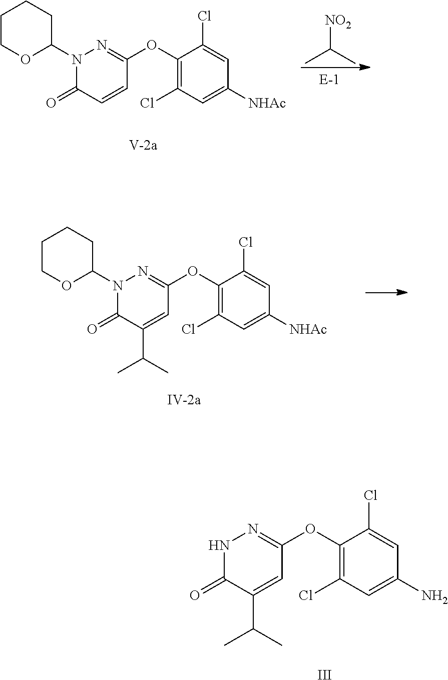

3.98 g of a compound V-2a (10 mmol), 1.34 g of a compound E-1 (15 mmol), 3.8 g of DBU (15 mmol), and 40 mL of DMSO were added to a reaction flask, and the resulting mixture was reacted at 50° C. with stirring for 12 h. The resulting reaction mixture was poured into 160 mL of water. The resulting system was adjusted with hydrochloric acid to a pH value of 6 and filtered to obtain a filter cake. The filter cake was washed once with 20 ml of water and oven-dried to obtain a compound IV-2a. 10 mL of a solution of hydrogen chloride in tetrahydrofuran was added to the compound IV-2a, and the resulting system was heated to 50° C. and reacted at 50° C. for 2 h. The resulting mixture was subjected to rotary evaporation until dryness. 20 mL of ethanol, 10 mL of water, and 2.4 g of sodium hydroxide (60 mmol) were added thereto, and the resulting system was heated to 100° C. and reacted at 100° C. for 5 h. The resulting reaction mixture was cooled to room temperature, and subjected to rotary evaporation to remove ethanol. 20 mL of ice water was added thereto, and the resulting system was adjusted with hydrochloric acid to a pH value of 6 such that a large amount of a solid was precipitated. The solid was separated through filtration, washed once with 20 mL of water, and then vacuum-dried to obtain a crude product. The crude product was subjected to recrystallization with acetonitrile and isopropyl ether to obtain 2.76 g of an off-white solid, i.e., compound III, with a yield of 88%. It was identified by nuclear magnetic resonance that the product had a target structure.

### Example 25 Preparation of a Compound Having a Structural Formula III

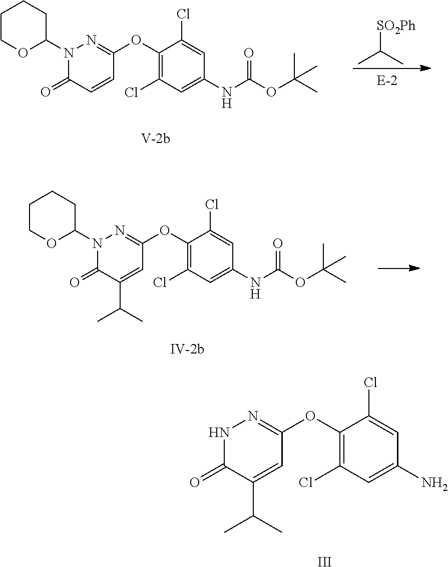

4.56 g of the compound V-2b (10 mmol), 2.2 g of a compound E-2 (12 mmol), 0.86 g of sodium amide (22 mmol), and 40 mL of DMF were added to a reaction flask, and the resulting mixture was subjected to a reaction at room temperature for 10 h. The resulting reaction mixture was poured into 120 mL of ice water. The resulting system was adjusted with hydrochloric acid to a pH value of 6 and filtered to obtain a filter cake. The filter cake was washed once with 20 mL of water and vacuum-dried to obtain a compound IV-2b. 15 mL of a solution of hydrogen chloride in 1,4-dioxane was added to the compound IV-2b, and the resulting system was heated to 60° C. and reacted at 60° C. for 2 h. The resulting reaction mixture was subjected to rotary evaporation until dryness, washed with 20 mL of a sodium bicarbonate solution, and vacuum-dried to obtain a crude product. The crude product was subjected to recrystallization with acetonitrile and isopropyl ether to obtain 2.64 g of an off-white solid, i.e., compound III, with a yield of 84%. It was identified by nuclear magnetic resonance that the product had a target structure.

### Example 26 Preparation of a Compound Having a Structural Formula III

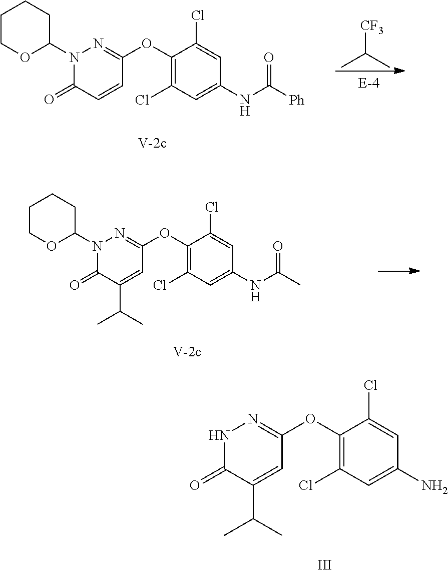

4.60 g of the compound V-2c (10 mmol), 1.79 g of a compound E-4 (16 mmol), 0.72 g of sodium hydride (30 mmol), and 40 mL of DMSO were added to a reaction flask, and the resulting mixture was subjected to a reaction at 60° C. for 6 h. The resulting reaction mixture was poured into 160 mL of ice water. The resulting system was adjusted with hydrochloric acid to a pH value of 6 and filtered to obtain a filter cake. The filter cake was washed once with 20 mL of water, then washed with 20 mL of a solvent consisting of ethyl acetate and heptane in a ratio of 1:2, and oven-dried to obtain a compound IV-2c. 20 mL of a solution of hydrogen chloride in tetrahydrofuran was added to the compound IV-2c, and the resulting mixture was heated to 55° C. and reacted at 55° C. for 2 h. The resulting reaction mixture was subjected to rotary evaporation until dryness. 20 mL of ethanol, 10 mL of water, and 2.4 g of sodium hydroxide (60 mmol) were added thereto, and the resulting mixture was heated to 100° C. and reacted at 100° C. for 5 h. The resulting reaction mixture was cooled to room temperature, and subjected to rotary evaporation to remove ethanol. 20 mL of ice water was added thereto, and the resulting system was adjusted with hydrochloric acid to a pH value of 6 such that a large amount of a solid was precipitated. The solid was separated through filtration, washed once with 20 ml of water, and then vacuum-dried to obtain a crude product. The crude product was subjected to recrystallization with acetonitrile and isopropyl ether to obtain 2.61 g of an off-white solid, i.e., compound III, with a yield of 83%. It was identified by nuclear magnetic resonance that the product had a target structure.

### Example 27 Preparation of a Compound Having a Structural Formula III

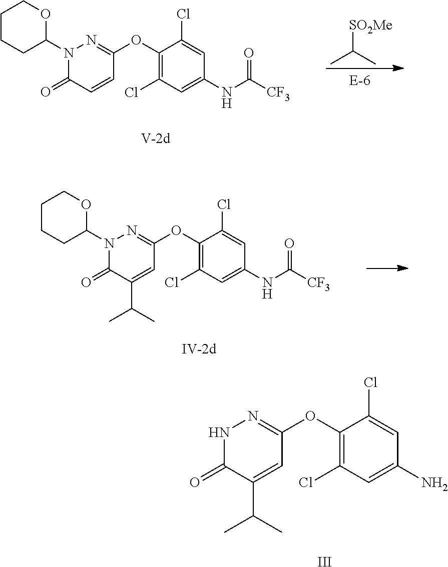

4.52 g of the compound V-2d (10 mmol), 1.95 g of a compound E-6 (16 mmol), 1.01 g of sodium amide (26 mmol), and 40 mL of DMF were added to a reaction flask, and the resulting mixture was subjected to a reaction at 60° C. for 6 h. The resulting reaction mixture was poured into 160 mL of ice water. The resulting system was adjusted with hydrochloric acid to a pH value of 6 and filtered to obtain a filter cake. The filter cake was washed once with 20 mL of water, then washed with 20 mL of a solvent consisting of ethyl acetate and heptane in a ratio of 1:2, and oven-dried to obtain a compound IV-2d. 20 mL of a solution of hydrogen chloride in tetrahydrofuran was added to the compound IV-2d. The resulting system was heated to 60° C. and reacted at 60° C. for 2 h, and subjected to rotary evaporation until dryness. 20 mL of ethanol, 10 ml of water, and 2.4 g of sodium hydroxide (60 mmol) were added thereto, and the resulting system was heated to 70° C. and reacted at 70° C. for 3 h. The resulting reaction mixture was cooled to room temperature, and subjected to rotary evaporation to remove ethanol. 20 mL of ice water was added thereto, and the resulting system was adjusted with hydrochloric acid to a pH value of 6 such that a large amount of a solid was precipitated. The solid was separated through filtration, washed once with 20 mL of water, and then vacuum-dried to obtain a crude product. The crude product was subjected to recrystallization with acetonitrile and isopropyl ether to obtain 2.67 g of an off-white solid, i.e., compound III, with a yield of 85%. It was identified by nuclear magnetic resonance that the product had a target structure.

### Example 28 Preparation of a Compound Having a Structural Formula III

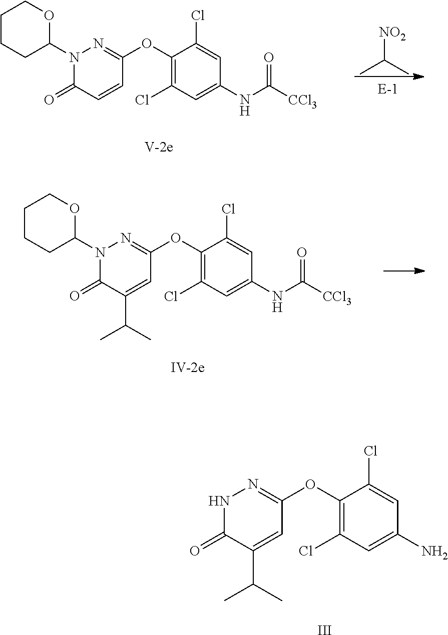

5.02 g of the compound V-2e (10 mmol), 1.16 g of a compound E-1 (13 mmol), 3.8 g of DBU (15 mmol), and 40 mL of DMSO were added to a reaction flask, and the resulting mixture was reacted at 60° C. with stirring for 8 h. The resulting reaction mixture was poured into 160 mL of water. The resulting system was adjusted with hydrochloric acid to a pH value of 6 and filtered to obtain a filter cake. The filter cake was washed once with 20 mL of water and oven-dried to obtain a compound IV-2e. 15 mL of a solution of hydrogen chloride in ethyl ether was added to the compound IV-2e. The resulting system was heated to 40° C. and reacted at 40° C. for 4 h, and subjected to rotary evaporation until dryness. 20 mL of methanol, 10 mL of water, and 2.4 g of sodium hydroxide (60 mmol) were added thereto, and the resulting mixture was heated to 70° C. and reacted at 70° C. for 3 h. The resulting reaction mixture was cooled to room temperature, and subjected to rotary evaporation to remove methanol. 20 mL of ice water was added thereto, and the resulting system was adjusted with hydrochloric acid to a pH value of 6 such that a large amount of a solid was precipitated. The solid was separated through filtration, washed once with 20 mL of water, and then vacuum-dried to obtain a crude product. The crude product was subjected to recrystallization with acetonitrile and isopropyl ether to obtain 2.83 g of an off-white solid, i.e., compound III, with a yield of 90%. It was identified by nuclear magnetic resonance that the product had a target structure.

### Example 29 Preparation of a Compound Having a Structural Formula III

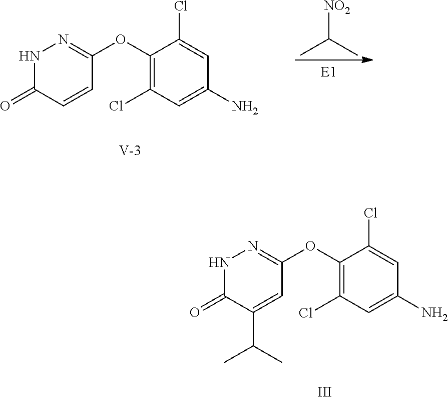

2.72 g of the compound V-3 (10 mmol), 1.07 g of a compound E-1 (12 mmol), 2.3 g of 1,1,3,3-tetramethylguanidine (20 mmol), and 30 mL of sulfolane were added to a reaction flask, and the resulting mixture was subjected to a reaction at 60° C. for 20 h. The resulting reaction mixture was poured into 120 mL of ice water to obtain a system. The system was adjusted with hydrochloric acid to a pH value of 6 and filtered to obtain a filter cake. The filter cake was washed once with 20 mL of water and vacuum-dried to obtain a crude product. The crude product was subjected to recrystallization with acetonitrile and isopropyl ether to obtain 2.45 g of an off-white solid, i.e., compound III, with a yield of 78%. It was identified by nuclear magnetic resonance that the product had a target structure.

### Example 30 Preparation of a Compound Having a Structural Formula III

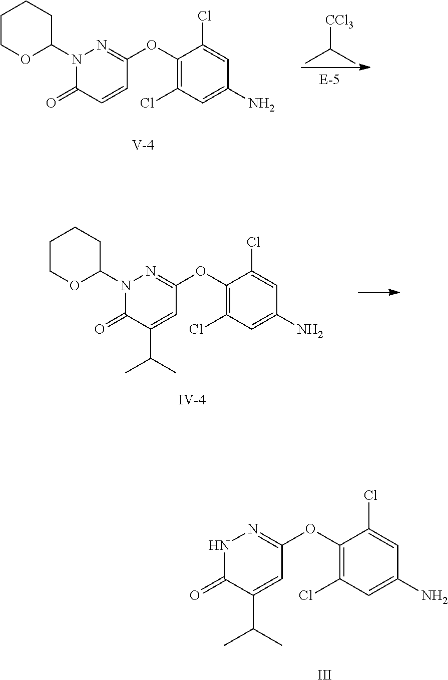

3.56 g of the compound V-4 (10 mmol), 2.42 g of a compound E-5 (15 mmol), 0.78 g of sodium amide (20 mmol), and 30 mL of DMAc were added to a reaction flask, and the resulting mixture was subjected to a reaction at 60° C. for 8 h. The resulting reaction mixture was poured into 160 mL of ice water. The resulting system was adjusted with hydrochloric acid to a pH value of 6 and filtered to obtain a filter cake. The filter cake was washed once with 20 ml of water and vacuum-dried to obtain a compound IV-4. 15 mL of a solution of hydrogen chloride in 1,4-dioxane was added to the compound IV-4, and the resulting mixture was heated to 50° C. and reacted at 50° C. for 2 h. The resulting reaction mixture was subjected to rotary evaporation until dryness, then washed with 20 mL of a sodium bicarbonate solution, and vacuum-dried to obtain a crude product. The crude product was subjected to recrystallization with acetonitrile and isopropyl ether to obtain 2.57 g of an off-white solid, i.e., compound III, with a yield of 82%. It was identified by nuclear magnetic resonance that the product had a target structure.

### Example 31 Preparation of a Compound Having a Structural Formula I

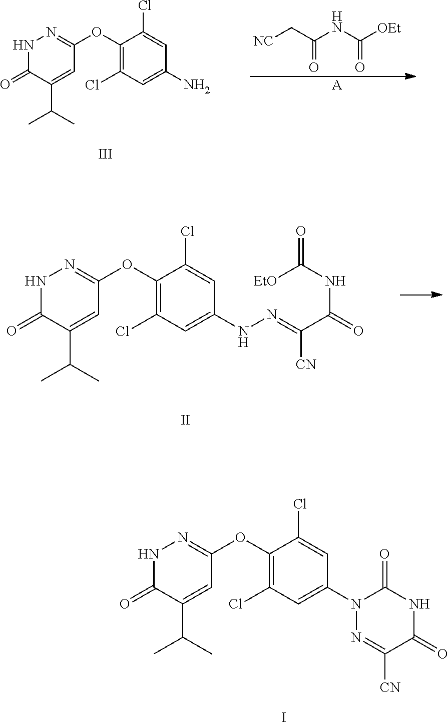

Preparation of a compound II: 3.14 g of the compound III (10.0 mmol), 1.72 g of a compound A (11.0 mmol), and 60 mL of water were added to a reaction flask and the resulting mixture was cooled in an ice water bath to a temperature of 0° C. to 10° C. 30 mL of hydrochloric acid (with a mass fraction of 37%) was added in batches. At a temperature of 5° C. to 10° C., a solution prepared from 6.9 g of sodium nitrite (100 mmol) and 20 mL of water was added dropwise thereto within 30 min, and then a solution prepared from 23.0 g of sodium acetate (280 mmol) and 90 mL of water was added dropwise thereto within 50 min. The ice water bath was removed, and the resulting mixture was stirred at room temperature and reacted for 2 h. The resulting reaction mixture was filtered to obtain a filter cake. The filter cake was washed with 30 mL of water, then washed with 20 mL of isopropyl ether, and oven-dried to obtain 4.23 g of a yellow-brown solid, i.e., compound II, with a yield of 88%, ¹H-NMR (400M, DMSO-d₆): δ 12.26 (2H, brs), 10.90 (1H, s), 8.00 (2H, s), 7.36 (1H, s), 4.23 (2H, q), 3.07 (1H, m), 1.28-1.20 (9H, m).

Preparation of a compound I: 2.41 g of the compound II (5.0 mmol), 0.54 g of potassium acetate (5.5 mmol), and 20 mL of DMF were added to a reaction flask, and the resulting mixture was heated to 110° C. and reacted at 110° C. for 3 h. The resulting reaction mixture was cooled to 80° C., and 1.0 mL of glacial acetic acid was added thereto. The resulting mixture was reacted for 2 h. The resulting reaction mixture was cooled to room temperature, and 100 mL of water was added thereto. The resulting system was adjusted with potassium carbonate to a pH value of 7, stirred for 5 min, and filtered to obtain a filter cake. The filter cake was washed with 20 mL of a solvent consisting of acetonitrile and water in a ratio of 1:2, and then oven-dried to obtain 1.96 g of a yellow-brown solid, i.e., compound I, with a yield of 90%, ¹H-NMR (400M, DMSO-d₆): 13.21 (1H, brs), 12.26 1H, brs), 7.81 (2H, s), 7.46 (1H, s), 3.06 (1H, m), 1.18 (6H, d).

The above are merely preferred embodiments of the present disclosure. It should be noted that a person of ordinary skill in the art may further make several improvements and modifications without departing from the principle of the present disclosure, but such improvements and modifications should be deemed as falling within the scope of the present disclosure.

What is claimed is:

1. A method for preparing a key intermediate III for Resmetirom, comprising:
providing a compound having a structure shown in formula V: 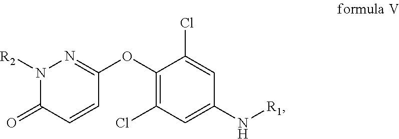
wherein in formula V, R₁ is H or an amino-protecting group, the amino-protecting group having 1 to 10 carbon atoms; and R₂ is H or 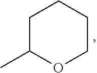
under the condition that R₁ and R₂ in formula V both are H, the method for preparing the key intermediate III for the Resmetirom further comprises:
(i) mixing the compound having the structure shown in formula V, a compound having a structure shown in formula E, an alkaline compound, and an organic solvent, and subjecting a resulting mixture to an addition-elimination-double bond migration reaction, to obtain the key intermediate III for the Resmetirom, the key intermediate III for the Resmetirom having a structural formula shown in formula III, 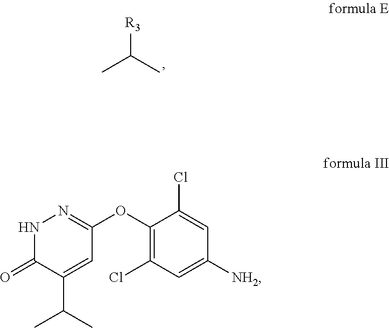
wherein in formula E, R₃ is selected from the group consisting of —NO₂, —SO₂Ph, —SO₂Me, —CN, —CF₃, and —CCl₃; and
under the condition that R₁ and R₂ in formula V are not both H, the method for preparing the key intermediate III for the Resmetirom further comprises:
(1) mixing the compound having the structure shown in formula V, a compound having the structure shown in formula E, an alkaline compound, and an organic solvent, and subjecting a resulting mixture to an addition-elimination-double bond migration reaction, to obtain a compound having a structure shown in formula IV, 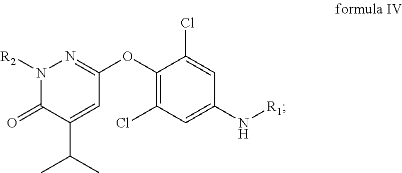
and
(2) removing R₁ and/or R₂ in the compound having the structure shown in formula IV through a deprotection reaction, to obtain the key intermediate III for the Resmetirom.

2. The method as claimed in claim 1, wherein the amino-protecting group is selected from the group consisting of t-BuOCO—, PhCO—, CH₃CO—, CF₃CO—, and CCl₃CO—.

3. The method as claimed in claim 1, wherein in step (i) and step (1), the alkaline compound is independently at least one selected from the group consisting of 1,8-diazabicyclo[5.4.0]undec-7-ene (DBU), 1,1,3,3-tetramethylguanidine, sodium amide, potassium tert-butoxide, sodium hydride, potassium carbonate, sodium carbonate, KOH, and NaOH; and a molar ratio of the compound having the structure shown in formula V to the alkaline compound independently ranges from 1:0.5 to 1:10;
in step (i) and step (1), a molar ratio of the compound having the structure shown in formula V to the compound having the structure shown in formula E independently ranges from 1:1 to 1:5; and
in step (i) and step (1), the addition-elimination-double bond migration reaction is conducted at a temperature independently of 0° C. to 120° C. for 1 h to 50 h independently.

4. The method as claimed in claim 2, wherein under the condition that in formula IV, R₁ is t-BuOCO— and R₂ is H or 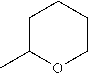
or under the condition that in formula IV, R₁ is H and R₂ is not H, the deprotection reaction in step (2) is an acidic deprotection reaction, and an acid adopted for the acidic deprotection reaction is selected from the group consisting of a solution of hydrogen chloride in ethyl ether, a solution of hydrogen chloride in tetrahydrofuran, and a solution of hydrogen chloride in 1,4-dioxane; and the acidic deprotection reaction is conducted at a temperature of 0° C. to 100° C. for 1 h to 10 h.

5. The method as claimed in claim 4, wherein in the solution of hydrogen chloride in ethyl ether, the solution of hydrogen chloride in tetrahydrofuran, or the solution of hydrogen chloride in 1,4-dioxane, a mass fraction of the hydrogen chloride independently ranges from 10% to 50%; and a dosage ratio of the compound having the structure shown in formula IV to the acid is in a range of 1 g: 2 mL to 1 g: 20 mL.

6. The method as claimed in claim 2, wherein under the condition that in formula IV, R₁ is not H or t-BuOCO— and R₂ is H, the deprotection reaction in step (2) is an alkaline hydrolysis reaction, and an alkali adopted for the alkaline hydrolysis reaction is selected from the group consisting of sodium hydroxide and potassium hydroxide; a molar ratio of the compound having the structure shown in formula IV to the alkali ranges from 1:2 to 1:10; a solvent adopted for the alkaline hydrolysis reaction is an organic solvent-water mixed solvent, and an organic solvent in the organic solvent-water mixed solvent is at least one selected from the group consisting of ethanol, methanol, tetrahydrofuran, 1,4-dioxane, and dimethyl sulfoxide (DMSO); and the alkaline hydrolysis reaction is conducted at a temperature of 30° C. to 130° C. for 2 h to 20 h.

7. The method as claimed in claim 2, wherein under the condition that in formula IV, neither R₁ nor R₂ is H and R₁ is not t-BuOCO—, the deprotection reaction in step (2) comprises an acidic deprotection reaction and an alkaline hydrolysis reaction that are conducted sequentially; an acid adopted for the acidic deprotection reaction is one selected from the group consisting of a solution of hydrogen chloride in ethyl ether, a solution of hydrogen chloride in tetrahydrofuran, and a solution of hydrogen chloride in 1,4-dioxane; the acidic deprotection reaction is conducted at a temperature of 0° C. to 100° C. for 1 h to 10 h; an alkali adopted for the alkaline hydrolysis reaction is selected from the group consisting of sodium hydroxide and potassium hydroxide; a molar ratio of the compound having the structure shown in formula IV to the alkali ranges from 1:2 to 1:10; a solvent adopted for the alkaline hydrolysis reaction is an organic solvent-water mixed solvent, and an organic solvent in the organic solvent-water mixed solvent is at least one selected from the group consisting of ethanol, methanol, tetrahydrofuran, 1,4-dioxane, and DMSO; and the alkaline hydrolysis reaction is conducted at a temperature of 30° C. to 130° C. for 2 h to 20 h.

8. The method as claimed in claim 1, wherein under the condition that in formula V, R₁ is not H and R₂ is H, the compound having the structure shown in formula V is prepared by a process comprising:
mixing a compound having a structure shown in formula D, a compound having a structure shown in formula G, a first alkaline compound, and an organic solvent, and subjecting a resulting mixture to a substitution reaction, to obtain a compound having a structure shown in formula VI, 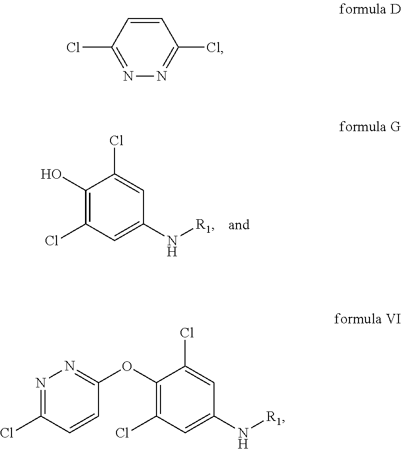
wherein R₁ in formula G and R₁ in formula VI are of the same type as R₁ in formula V; and
mixing the compound having the structure shown in formula VI, acetic acid, and sodium acetate, and subjecting a resulting mixture to a substitution-hydrolysis reaction, to obtain the compound having the structure shown in formula V, which has a structural formula shown in formula V-1,

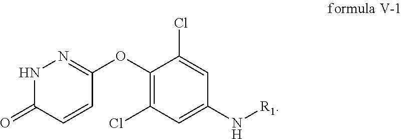

9. The method as claimed in claim 1, wherein under the condition that in formula V, R₁ is not H and R₂ is not H, the compound having the structure shown in formula V is prepared by a process comprising:
mixing a compound having a structure shown in formula V-1, 3,4-dihydro-2H-pyran, an acid, and an organic solvent, and subjecting a resulting mixture to an amide protection reaction, to obtain the compound having the structure shown in formula V, which has a structural formula shown in formula V-2, 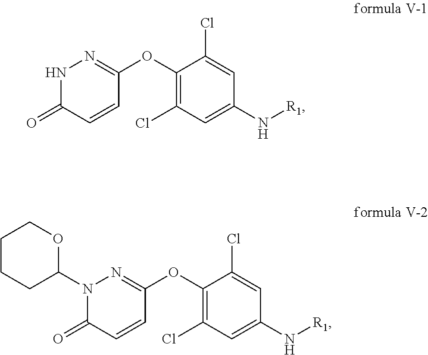
wherein in formula V-1, R₁ is an amino-protecting group.

10. The method as claimed in claim 1, wherein under the condition that in formula V, R₁ is H and R₂ is H, the compound having the structure shown in formula V is prepared by a process comprising:
mixing a compound having a structure shown in formula V-1, a second alkaline compound, an organic solvent, and water, and subjecting a resulting mixture to a first hydrolysis reaction, to obtain the compound having the structure shown in formula V, which has a structural formula shown in formula V-3: 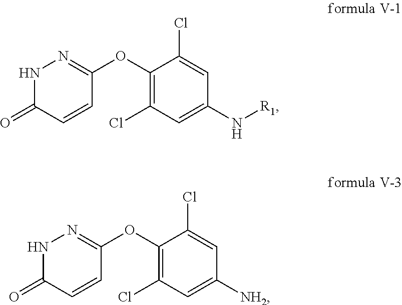
wherein in formula V-1, R₁ is selected from the group consisting of PhCO—, CF₃CO—, CCl₃CO—, and CH₃CO—.

11. The method as claimed in claim 1, wherein under the condition that in formula V, R₁ is H and R₂ is not H, the compound having the structure shown in formula V is prepared by a process comprising:
mixing a compound having a structure shown in formula V-2, a third alkaline compound, an organic solvent, and water, and subjecting a resulting mixture to a second hydrolysis reaction, to obtain the compound having the structure shown in formula V, which has a structural formula shown in formula V-4: 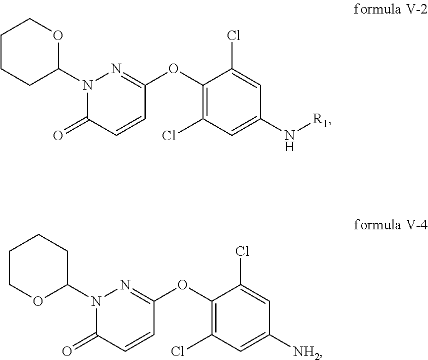
wherein in formula V-2, R₁ is selected from the group consisting of PhCO—, CF₃CO—, CCl₃CO—, and CH₃CO—.

12. The method as claimed in claim 8, wherein a molar ratio of the compound having the structure shown in formula D to the compound having the structure shown in formula G ranges from 1:1 to 1:2;
the first alkaline compound is at least one selected from the group consisting of an alkali metal carbonate, an alkali metal hydroxide, and an organic base, and a molar ratio of the compound having the structure shown in formula D to the first alkaline compound ranges from 1:1 to 1:3;
the organic solvent adopted for the substitution reaction is at least one selected from the group consisting of N,N-dimethylformamide (DMF), N,N-dimethylacetamide (DMAc), DMSO, N-methylpyrrolidone (NMP), acetonitrile, and 1,4-dioxane, and the substitution reaction is conducted at a temperature of 40° C. to 120° C. for 5 h to 50 h;
a molar ratio of the compound having the structure shown in formula VI to the sodium acetate ranges from 1:1 to 1:5, and a molar ratio of the compound having the structure shown in formula VI to the acetic acid ranges from 1:10 to 1:50; and
the substitution-hydrolysis reaction is conducted at a temperature of 60° C. to 150° C. for 2 h to 20 h.

13. The method as claimed in claim 9, wherein in the amide protection reaction, a molar ratio of the compound having the structure shown in formula V-1 to the acid ranges from 1:0.05 to 1:0.5, and a molar ratio of the compound having the structure shown in formula V-1 to the 3,4-dihydro-2H-pyran ranges from 1:1 to 1:4;
the organic solvent adopted for the amide protection reaction is at least one selected from the group consisting of tetrahydrofuran, ethyl acetate, dichloromethane, acetonitrile, acetone, and 1,4-dioxane; and
the amide protection reaction is conducted at a temperature of 30° C. to 80° C. for 1 h to 10 h.

14. The method as claimed in claim 10, wherein in the first hydrolysis reaction, a molar ratio of the compound having the structure shown in formula V-1 to the second alkaline compound ranges from 1:2 to 1:6, and the second alkaline compound is an alkali metal hydroxide;
the organic solvent adopted for the first hydrolysis reaction is at least one selected from the group consisting of ethanol, methanol, tetrahydrofuran, and DMSO; and
the first hydrolysis reaction is conducted at a temperature of 50° C. to 120° C. for 1 h to 10 h.

15. The method as claimed in claim 11, wherein in the second hydrolysis reaction, a molar ratio of the compound having the structure shown in formula V-2 to the third alkaline compound ranges from 1:2 to 1:6, and the third alkaline compound is an alkali metal hydroxide;
the organic solvent adopted for the second hydrolysis reaction is at least one selected from the group consisting of ethanol, methanol, tetrahydrofuran, and DMSO; and
the second hydrolysis reaction is conducted at a temperature of 50° C. to 120° C. for 1 h to 10 h.
